(19) United States
(12) Patent Application Publication
(10) Pub. No.: US 2020/0190581 A1
(43) Pub. Date: Jun. 18, 2020

(54) METHODS FOR DETECTING CYTOSINE MODIFICATIONS

(71) Applicant: The University of Chicago, Chicago, IL (US)

(72) Inventors: Chuan HE, Chicago, IL (US); Xingyu LU, Chicago, IL (US); Lulu HU, Chicago, IL (US)

(21) Appl. No.: 16/475,402

(22) PCT Filed: Jan. 4, 2018
(86) PCT No.: PCT/US18/12288

§ 371 Date: Jul. 2, 2019

Related U.S. Application Data
(60) Provisional application No. 62/442,230, filed on Jan. 4, 2017.

(51) Int. Cl. C12Q 1/6876; C12N 15/11

(52) U.S. Cl. CPC C12Q 1/6876; C12N 2310/531; C12Q 2600/154; C12N 15/11

(57) ABSTRACT

The current disclosure provides a method that can specifically label and directly amplify 5hmC site on genomic DNA without pull-down or bisulfite treatment, which enables one to map the 5hmC site from a single DNA molecule. Aspects of the disclosure relate to a method for detecting 5-hydroxymethylcytosine (5hmC) nucleic acid bases in a nucleic acid molecule or a plurality of nucleic acid molecules, the method comprising: a. modifying the 5hmC nucleic acid base with a first functional group; b. covalently attaching a modified nucleic acid probe comprising a second functional group to the first functional group; wherein the nucleic acid probe and nucleic acid molecule are covalently linked through the first and second functional groups; c. annealing a primer to the nucleic acid probe; d. performing primer extension of the annealed primer to make a new strand; and e. detecting the new strand.

### CROSS-REFERENCE TO RELATED APPLICATIONS

This application claims the benefit of priority of U.S. Provisional Patent Application No. 62/442,230 filed Jan. 4, 2017, which is hereby incorporated by reference in its entirety.

### STATEMENT OF GOVERNMENT SUPPORT

The invention was made with government support under grant no.: R01 HG006827 awarded by National Institutes of Health. The government has certain rights in the invention.

### BACKGROUND OF THE INVENTION

### I. Field of the Invention

Embodiments of this invention are directed generally to cell biology. In certain aspects methods involve determining whether 5-methycytosine and/or 5-hydroxymethylcytosine is present in a nucleic acid molecule.

### II. Background

5-methylcytosine (5mC) and 5-hydroxymethylcytosine (5hmC) are important epigenetic markers in mammalian cells. Current 5mC and 5hmC sequencing methods can be summarized as: 1) bisulfite conversion-based methods; 2) affinity capture-based methods including antibody-based pull-down and selective chemical labeling-based pull-down; 3) restriction endonuclease-based methods. All these existing methods require micro-grams of input genomic DNA. The large quantity of input limits the research application for rare samples and single cell systems, such as single cell behaviors during differentiation. Bisulfite conversion-based methods are considered to be the gold standard due to its ability to quantitatively differentiate 5mC and normal C in single-base resolution. However, DNA degradation is a major drawback. Affinity-based methods are relatively inexpensive but have low resolution and may lose information for low CpG density coverage (antibody-based methods). Restriction endonuclease methods have limited resolution and the coverage depends on the sequence specificity and methylation or hydroxylmethyaltion sensitivity. Overall, none of the current methods can sequence 5mC and 5hmC in small amount of DNA (nano-gram scale or sub nano-gram scale) or obtain information for these modifications in single cell level. Therefore, there is a need in the art for more methods for detecting cytosine modifications such as 5mC and 5hmC in small amounts of DNA.

### SUMMARY OF THE INVENTION

The currend disclosure fulfulls the aforementioned need in the art by providing a method, referred to as Jump-seq, that can specifically label and directly amplify 5hmC site on genomic DNA without pull-down or bisulfite treatment, which enables one to map the 5hmC site from a single DNA molecule. Aspects of the disclosure relate to compositions and methods for detecting 5-hydroxymethylcytosine (5hmC); detecting 5-methylcytosine (5-mC); distinguishing 5hmC from cytosine, 5-mC, or another cytosine modification; distinguishing 5mC from cytosine, 5-hmC, or another cytosine modification; identifying 5-hmC; identifying 5-mC; mapping 5-hmC; mapping 5-mC; locating 5-hmC; locating 5-mC; quantifying 5-hmC; and, quantifying 5-mC. Any of the steps disclosed herein may be employed for these methods, and kits or compositions may include one or more components disclosed herein.

In some embodiments, there is a method for detecting 5-hydroxymethylcytosine (5hmC) nucleic acid bases in a nucleic acid molecule or a plurality of nucleic acid molecules, the method comprising: one or more or all of the following steps: a) modifying the 5hmC nucleic acid base with a first functional group; b) covalently attaching a modified nucleic acid probe comprising a second functional group to the first functional group; wherein the nucleic acid probe and nucleic acid molecule are covalently linked through the first and second functional groups; c) annealing a primer to the nucleic acid probe; d) performing primer extension of the annealed primer to make a new strand; and e) detecting the new strand.

Further aspects relate to a method for detecting 5-methylcytosine (5-mC) nucleic acid bases in a nucleic acid molecule or a plurality of nucleic acid molecules, the method comprising one or more or all of the following steps: a) modifying 5hmC nucleic acid bases with a glucose molecule; b) oxidizing 5-mC to 5-hmC to make converted 5hmC; c) modifying the converted 5-hmC nucleic acid base with a first functional group; d) covalently attaching a modified nucleic acid probe comprising a second functional group to the first functional group; wherein the nucleic acid probe and nucleic acid molecule are covalently linked through the first and second functional groups; e) annealing a primer to the nucleic acid probe; f) performing primer extension of the annealed primer to make a new strand; and g) detecting the new strand.

Methods may include any of the steps identified herein; embodiments may also include separating or purifying one or more components of a reaction, such as a reaction product. Certain embodiments are directed to methods for detecting 5mC in a nucleic acid comprising converting 5mC to a modified 5mC, such as 5-hydroxymethylcytosine and detecting 5-hydroxymethylcytosine. In certain aspects, the 5-methylcytosine is converted to 5-hydroxymethylcytosine using enzymatic modification by a methylcytosine dioxygenase or the catalytic domain of a methylcytosine dioxygenase. In a further aspect, a methylcytosine dioxygenase is TET1, TET2, or TET3, or a homolog thereof.

In some embodiments, the nucleic acid probe is covalently linked to the second functional group. In some embodiments, the nucleic acid probe comprises at least, at most, or exactly 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 5, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, or 150 nucleotides (or any derivable range therein). In some embodiments, the second functional group is covalently linked to the 5′ or 3′ end of the nucleic acid. In some embodiments, the second functional group is covalently linked to the 5′ end of the nucleic acid. In some embodiments, the second functional group is covalently linked to the 3′ end of the nucleic acid. In some embodiments, the nucleic acid probe comprises a primer annealing region where a primer may bind through complementary base pairing. In some embodiments, there at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides (or any derivable range therein) between the primer annealing region and the second functional group. In some embodiments, the primer annealing region is at least, at most, or exactly 7, 8, 9, 10, 11, 12, 13, 14, 15, 16, 17, 18, 19, 20, 21, 22, 23, 24, 25, 26, 27, 28, 29, or 30 nucleotides in length (or any derivable range therein).

In some embodiments, detecting the new strand comprises sequencing the new strand. In some embodiments, detecting the new strand comprises polymerase chain reaction (PCR). In some embodiments, the PCR is quantitative PCR.

In some embodiments, the primer and/or probe is labeled with one or more detection moieties. In some embodiments, the newly synthesized strands are labeled with one or more detection moieties. In some embodiments, the detection moiety comprises a fluorescent molecule. In some embodiments, the detection moiety/label is one described herein. In some embodiments, detecting the new strand comprises detecting the detection moiety.

In some embodiments, the methods comprise the use of an array. In some embodiments, the new strand is annealed to an array comprising nucleic acids. In some embodiments in which the new strand is labeled one or more detection moieties, the new strands may be annealed to a nucleic acid array, and the label may be detected to quantitatively or qualitatively determine the abundance of a specific loci in the newly synthesized strand population.

In some embodiments, the nucleic acid molecule comprises DNA. In some embodiments, the DNA is genomic DNA. In some embodiments, the nucleic acid molecule comprises RNA. In some embodiments, the nucleic acid comprises cell free DNA. In some embodiments, the cell-free DNA is isolated from a biological sample such as blood, a stool sample, a saliva sample, a tissue sample, etc. In some embodiments, the nucleic acid is isolated from a tissue sample. In some embodiments, the nucleic acid is isolated from a biopsy sample. In particular embodiments, the nucleic acid molecule is isolated, such as away from non-nucleic acid cellular material and/or away from other nucleic acid molecules.

In some embodiments, the first functional group is covalently attached to a glucose or a modified glucose molecule. In some embodiments, the 5hmC is modified with a glucose or a modified glucose molecule. In some embodiments, modifying the 5hmC nucleic acid base with a glucose or a modified glucose comprises incubating the nucleic acid molecule with a β-glucosyltransferase and a glucose or modified glucose molecule. In some embodiments, the modified glucose molecule is uridine diphospo6-N₃-glucose molecule.

In some embodiments, performing primer extension of the annealed primer to make a new strand comprises contacting the nucleic acid with a polymerase. Methods of primer extension are known in the art.

In some embodiments, the first or second functional groups comprise an alkyne or azide. In further embodiments, the first or second functional groups comprise a compatible functional pair as described herein. In some embodiments, the first and second functional groups are covalently linked using Click Chemistry. In some embodiments, the first or second functional groups comprise a thiol or maleimide.

In some embodiments, the nucleic acid probe is modified with a molecule having a molecular mass or weight of at least 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 425, 450, 475, 500, 525, 550, 575, or 600 u, or any derivable range therein. In some embodiments, the molecule comprises dibenzocycloctyne (DBCO).

In some embodiments, the method further comprises cloning the new strand into a plasmid or expression construct.

In some embodiments, sequencing the new strand comprises sequencing by Sanger sequencing, Maxam-Gilbert sequencing, SOLiD sequencing, sequencing by synthesis, pyrosequencing, Ion Torrent semiconductor sequencing, massively parallel signature sequencing, polony sequencing, 454 pyrosequencing, Illumina dye sequencing, DNA nanoball sequencing, or single-molecule real-time sequencing. In some embodiments, the methods exclude bisulfite treatment of the nucleic acid.

In some embodiments, the method further comprises fragmenting the nucleic acid. In some embodiments, the method further comprises tagging the nucleic acid. In some embodiments, the nucleic acid is tagged and/or fragmented by a transposome. In some embodiments, tagging and/or fragmenting the nucleic acid comprises contacting the contacting the nucleic acid molecule with a transposase and a transposon. In some embodiments, the transposon comprises a P7 adapter-containing transposon. In some embodiments, the transposon comprises an affinity tag. In some embodiments, the affinity tag comprises biotin. In some embodiments, the transposon comprises an affinity tag as described herein.

In some embodiments, the method further comprises isolating or purifying the fragmented nucleic acid molecules by contacting the nucleic acid molecules with a capture reagent, wherein the capture reagent binds to the affinity tag; and separating the capture reagent bound to the affinity tagged fragmented nucleic acid molecules from surrounding components.

In some embodiments, the method further comprises sorting a population of cells into isolated single cells. The cells may be sorted by methods known in the art such as FACS or by serial dilutions of populations of cells. In some embodiments, the method further comprises tagging the nucleic acid of each single cell with a unique nucleic acid sequence. In some embodiments, the method further comprises pooling the tagged nucleic acids into a single composition.

In some embodiments, the method further comprises end repair of the nucleic acid. End repair kits are known in the art and commercially available and can be used for the conversion of DNA containing damaged or incompatible 5′ and or 3′ protruding ends to 5′ phosphorylated, blunt-ended DNA. In some embodiments, the method further comprises ligation of an adaptor sequence onto the fragmented DNA.

In some embodiments, the primer is covalently attached to the nucleic acid probe. For example, the primer may be contiguous with the nucleic acid probe. In some embodiments, the primer is at least, at most, or exactly 7, 8, 9, 10, 11, 12, 13, 14, 15, 16, 17, 18, 19, 20, 21, 22, 23, 24, 25, 26, 27, 28, 29, or 30 nucleotides in length (or any derivable range therein). In some embodiments, the primer is at least, at most, or exactly 100, 99, 98, 97, 96, 95, 94, 93, 92, 91, 90, 89, 88, 87, 86, or 85% complementary (or any derivable range therein) to the primer annealing region of the nucleic acid probe. In some embodiments, the probe comprises a cleavage site. In some embodiments, the cleavage site comprises a restriction enzyme cleavage site. In some embodiments, the nucleic acid probe comprises a hairpin. In some embodiments, the hairpin comprises a loop and wherein the loop comprises deoxyribose uracils. In some embodiments, the loop region comprises at least, at most, or exactly 1, 2, 3, 4, 5, 6, 7, 8, 9, 10, 12, or 14 or more deoxyribose uracils (or any derivable range therein). In some embodiments, the loop comprises at least three deoxyribose uracils. In some embodiments, the loop region comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides (or any derivable range therein). In some embodiments, the method further comprises cleaving the loop with a uracil DNA glycosylase. In some embodiments, the uracil DNA glycosylase comprises a USER™ enzyme. In some embodiments, the probe and/or primer further comprises a P5 adapter. In some embodiments, the second functional group is attached to the 5′ end of the nucleic acid probe.

In some embodiments, the method further comprises denaturing the nucleic acid molecule after step (d) and prior to step (e). In some embodiments, denaturing the nucleic acid comprises heating the nucleic acid to at least 70° C. In some embodiments, denatureing the nucleic acid comprises heating the nucleic acid to at least, at most, or exactly about 65, 70, 75, 80, 85, 90, 95, 100, 105, or 110° C., or any derivable range therein. In some embodiments, the method further comprises amplifying the new strand by PCR. In some embodiments, the new strand is amplified using nucleic acid primers; wherein at least one of the nucleic acid primers corresponds to a sequence in the inserted transposon (or a complement thereof) and at least one of the nucleic acid primers corresponds to a sequence in the nucleic acid probe (or a complement thereof). In some embodiments wherein the new strand is amplified using nucleic acid primers, at least one of the nucleic acid primers corresponds to a known genomic sequence near a potential modification site (or a complement thereof) and at least one of the nucleic acid primers corresponds to a sequence in the nucleic acid probe (or a complement thereof). In this case, the method may detect modification at a particular known genomic site. The amplification primer may be from a genomic site near the suspected modification site (or a complement thereof). The other primer may be a sequence within the nucleic acid probe or complementary thereto. If the modification is present, the new strand is synthesized through primer extension and the two amplification primers are capable of amplifying the new strand. In some embodiments, the new strand is amplified before sequencing.

In some embodiments, the method is for detecting 5-hydroxymethylcytosine (5hmC) nucleic acid bases in a nucleic acid molecule or a plurality of nucleic acid molecules isolated from a biological sample from a subject. In some embodiments, the biological sample is a tissue sample. In some embodiments, the tissue sample is a biopsy sample. The tissue sample may be one that is suspected of having an abnormality or disease such as cancer. In certain embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects the sample is obtained from cystic fluid or fluid derived from a tumor or neoplasm. In yet other embodiments the cyst, tumor or neoplasm is colorectal. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, scraping, phlebotomy, or any other methods known in the art. In some cases, the sample may be obtained, stored, or transported using components of a kit of the present methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

In some embodiments, the nucleic acid molecule or molecules are present in an amount of less than 50 ng. In some embodiments, the nucleic acid molecule or molecules are present in an amount of less than, at most, or exactly 1000, 750, 500, 250, 225, 200, 175, 150, 125, 100, 75, 50, 45, 40, 35, 30, 25, 20, 15, 14, 13, 12, 11, 10, 9, 8, 7, 6, 5, 4, or 3 nanograms (or any derivable range therein).

A polypeptide is considered as a homologue to another polypeptide when two polypeptides have at least 75% sequence identity. In some embodiments, the sequence identity level is 80% or 85%, 90% or 95%, 98%, 99% or 100% (or any range derivable therein). Similarly, a polynucleotide is considered as a homologue to another polynucleotide when two polynucleotides have at least 75% sequence identity. In some embodiments, the sequence identity level is 80% or 85%, 90% or 95%, and 98% or 99% (or any range derivable therein).

Methods may involve any of the following steps described herein and in any particular order, unless indicated otherwise.

In some embodiments, methods may also involve one or more of the following regarding nucleic acids prior to and/or concurrent with 5mC modification of nucleic acids: obtaining nucleic acid molecules; obtaining nucleic acid molecules from a biological sample; obtaining a biological sample containing nucleic acids from a subject; isolating nucleic acid molecules; purifying nucleic acid molecules; obtaining an array or microarray containing nucleic acids to be modified; denaturing nucleic acid molecules; shearing or cutting nucleic acid; denaturing nucleic acid molecules; hybridizing nucleic acid molecules; incubating the nucleic acid molecule with an enzyme that does not modify 5mC; incubating the nucleic acid molecule with a restriction enzyme; attaching one or more chemical groups or compounds to the nucleic acid or 5mC or modified 5mC; conjugating one or more chemical groups or compounds to the nucleic acid or 5mC or modified 5mC; incubating nucleic acid molecules with an enzyme that modifies the nucleic acid molecules or 5mC or modified 5mC by adding or removing one or more elements, chemical groups, or compounds.

Methods may also involve the following steps: modifying or converting a 5mC to 5-hydroxymethylcytosine (5hmC); modifying 5hmC using β-glucosyltransferase (βGT); incubating β-glucosyltransferase with UDP-glucose molecules and a nucleic acid substrate under conditions to promote glycosylation of the nucleic acid with the glucose molecule (which may or may not be modified) and result in a nucleic acid that is glycosylated at one or more 5-hydroxymethylcytosines.

It is contemplated that some embodiments will involve steps that are done in vitro, such as by a person or a person controlling or using machinery to perform one or more steps.

Methods and compositions may involve a purified nucleic acid, modification reagent or enzyme, label, chemical modification moiety, modified UDP-Glc, and/or enzyme, such as β-glucosyltransferase. Such protocols are known to those of skill in the art.

In certain embodiments, purification may result in a molecule that is about or at least about 70, 75, 80, 85, 90, 95, 96, 97, 98, 99, 99.1, 99.2, 99.3, 99.4, 99.5, 99.6, 99.7 99.8, 99.9% or more pure, or any range derivable therein, relative to any contaminating components (w/w or w/v).

In other methods, there may be steps including, but not limited to, obtaining information (qualitative and/or quantitative) about one or more 5mCs and/or 5hmCs in a nucleic acid sample; ordering an assay to determine, identify, and/or map 5mCs and/or 5hmCs in a nucleic acid sample; reporting information (qualitative and/or quantitative) about one or more 5mCs and/or 5hmCs in a nucleic acid sample; comparing that information to information about 5mCs and/or 5hmCs in a control or comparative sample. Unless otherwise stated, the terms “determine,” “analyze,” “assay,” and “evaluate” in the context of a sample refer to chemical or physical transformation of that sample to gather qualitative and/or quantitative data about the sample. Moreover, the term “map” means to identify the location within a nucleic acid sequence of the particular nucleotide.

In some embodiments, nucleic acid molecules may be DNA, RNA, or a combination of both. Nucleic acids may be recombinant, genomic, or synthesized. In additional embodiments, methods involve nucleic acid molecules that are isolated and/or purified. The nucleic acid may be isolated from a cell or biological sample in some embodiments. Certain embodiments involve isolating nucleic acids from a eukaryotic, mammalian, or human cell. In some cases, they are isolated from non-nucleic acids. In some embodiments, the nucleic acid molecule is eukaryotic; in some cases, the nucleic acid is mammalian, which may be human. This means the nucleic acid molecule is isolated from a human cell and/or has a sequence that identifies it as human. In particular embodiments, it is contemplated that the nucleic acid molecule is not a prokaryotic nucleic acid, such as a bacterial nucleic acid molecule. In additional embodiments, isolated nucleic acid molecules are on an array. In particular cases, the array is a microarray. In some cases, a nucleic acid is isolated by any technique known to those of skill in the art, including, but not limited to, using a gel, column, matrix or filter to isolate the nucleic acids. In some embodiments, the gel is a polyacrylamide or agarose gel.

Methods and compositions may also involve one or more enzymes. In some embodiments, the enzyme is a polymerase. In certain cases, embodiments involve a restriction enzyme. The restriction enzyme may be methylation-insensitive. In certain embodiments, nucleic acids are contacted with a restriction enzyme prior to, concurrent with, or subsequent to modification of 5mC. The modified nucleic acid may be contacted with a polymerase before or after the nucleic acid probe has been covalently attached to the nucleic acid.

Methods and compositions involve detecting, characterizing, and/or distinguishing between methylcytosine after modifying the 5mC. Methods may involve identifying 5mC in the nucleic acids by comparing modified nucleic acids with unmodified nucleic acids or to nucleic acids whose modification state is already known. Detection of the modification can involve a wide variety of recombinant nucleic acid techniques. In some embodiments, a modified nucleic acid molecule is incubated with polymerase, at least one primer, and one or more nucleotides under conditions to allow polymerization of the modified nucleic acid. In additional embodiments, methods may involve sequencing a modified nucleic acid molecule. In other embodiments, a modified nucleic acid is used in a primer extension assay.

Methods and compositions may involve a control nucleic acid. The control may be used to evaluate whether modification or other enzymatic or chemical reactions are occurring. Alternatively, the control may be used to compare modification states. The control may be a negative control or it may be a positive control. It may be a control that was not incubated with one or more reagents in the modification reaction. Alternatively, a control nucleic acid may be a reference nucleic acid, which means its modification state (based on qualitative and/or quantitative information related to modification at 5mCs, or the absence thereof) is used for comparing to a nucleic acid being evaluated. In some embodiments, multiple nucleic acids from different sources provide the basis for a control nucleic acid. Moreover, in some cases, the control nucleic acid is from a normal sample with respect to a particular attribute, such as a disease or condition, or other phenotype. In some embodiments, the control sample is from a different patient population, a different cell type or organ type, a different disease state, a different phase or severity of a disease state, a different prognosis, a different developmental stage, etc.

Embodiments also concern kits, which may be in a suitable container, that can be used to achieve the described methods. In certain embodiments, kits are provided for converting 5mC to 5hmC, modifying 5hmC of nucleic acid and/or subject such modified nucleic acid for further analysis, such as mapping 5mC or sequencing the nucleic acid molecule.

In certain aspect, the contents of a kit can include a methylcytosine dioxygenase, or its homologue and a 5-hydroxymethylcytosine modifying agent. In further aspects, the methylcytosine dioxygenase is TET1, TET2, or TET3. In other embodiments the kit includes the catalytic domain of TET1, TET2, or TET3. In certain aspects, the 5hmC modifying agent, which refers to an agent that is capable of modifying 5hmC, is β-glucosyltransferase.

In additional embodiments, a kit also contains a 5hmC modification, such as uridine diphophoglucose or a modified uridine diphophoglucose molecule. In particular embodiments, the modified uridine diphosphoglucose molecule can be uridine diphospho6-N₃-glucose molecule. In additional embodiments, a kit may also contain biotin.

Certain embodiments are directed to kits comprising a vector comprising a promoter operably linked to a nucleic acid segment encoding a methylcytosine dioxygenase or a portion and a 5-hydroxymethylcytosine modifying agent. In certain aspects, the nucleic segment encodes TET1, TET2, or TET3, or their catalytic domain. In certain aspects, the 5hmC modifying agent is β-glucosyltransferase. In additional aspects, a kit also contains a 5hmC modification, such as uridine diphophoglucose or a modified uridine diphophoglucose molecule. In particular embodiments, the modified uridine diphosphoglucose molecule can be uridine diphospho6-N₃-glucose molecule. In additional embodiments, a kit may also contain biotin.

In some embodiments, there are kits comprising one or more modification agents (enzymatic or chemical) and one or more modification moieties. The molecules may have or involve different types of modifications. In further embodiments, a kit may include one or more buffers, such as buffers for nucleic acids or for reactions involving nucleic acids. Other enzymes may be included in kits in addition to or instead of β-glucosyltransferase. In some embodiments, an enzyme is a polymerase. Kits may also include nucleotides for use with the polymerase. In some cases, a restriction enzyme is included in addition to or instead of a polymerase. In some embodiments, the kits include a nucleic acid probe. The nucleic acid probe may or may not already be modified. In some embodiments, the kits include modification moieties for attaching to the nucleic acid probe.

Other embodiments also concern an array or microarray containing nucleic acid molecules that have been modified at the nucleotides that were 5hmC and/or 5mC.

The following patent applications describe embodiments useful in the methods of the current disclosure: WO2011127136, WO2012138973, and WO2014165770, which are herein incorporated by reference.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B (A) 5hmC in genomic DNA is labeled with an azide-modified glucose using β-GT. 5mC is oxidized into 5hmC with Tet-coupled oxidation and then labeled with the use of β-GT. A hairpin DNA (with P5 adapter sequence) carrying an alkyne is added covalently to the modified glucose. (B) Genomic DNA is fragmented and tagged with P7 adapter sequence by transposase, followed by 5mC/5hmC labeling. After primer extension from the hairpin and cleavage from the tethered hairpin, the newly synthesized strand can be subjected to library construction and sequencing. 5mC/5hmC single sites can be inferred from the polymerase “landing” site pattern that connects the hairpin sequence and any genomic DNA sequence.

FIG. 2A-D. Reads distribution of Jump-seq Strategy. Preliminary Jump-seq results performed on genomic DNA isolated from 400 (2.4 ng), 1000 (6 ng), 2000 (12 ng), 4000 (24 ng), 8000 (48 ng) mouse ES cells showing a base-resolution “valley” of 5mC/5hmC overlaid on top of the 5mC/5hmC sites. “0” means the exact 5mC or 5hmC site. (A) 5mC-Jump-seq minus stand methyl sites (Jump-mC−). (B) 5mC-Jump-seq plus stand methyl sites (Jump-mC+). (C) 5hmC-Jump-seq minus stand hydroxymethyl sites (Jump-hmC−).(D) 5hmC-Jump-seq plus stand hydroxymethyl sites (Jump-hmC+). Noting that the Jump-seq strategy has a complementary strand synthesis step, therefore the reads mapped to the plus stand actually represent the mC/hmC sites in minus strand. That also applies to reads mapped to the minus strand.

FIG. 3. Single cell 5mC/5hmC Jump-seq Strategy. Target cells are sorted from a heterogeneous mixture of cells into 384 well plate in a one-cell-one-well manner based on the specific fluorescent signals. Sorted single cells are fragmented, pre-indexed and P7 tagged by barcoded transposomes and then pooled together in one tube, followed by Jump-seq treatment and Next-Generation Sequencing.

FIG. 4. Single cell 5mC/5hmC-Seal Strategy. Sorted single cells are fragmented, pre-indexed and P5 tagged by barcoded transposomes and then pooled together in one tube, followed by P7 ligation, azide-Glucose installation, biotin labeling. Then 5mC/5hmC containing DNA fragments are specifically enriched by streptavidin beads for library construction and next-generation sequencing.

FIG. 5. Cell free DNA 5mC/5hmC Jump-seq Strategy. Cell free DNA is end repaired, ligated with biotin labeled P7 followed by ordinary 5mC/5hmC Jump-seq.

FIG. 6 shows exemplary molecules that the nucleic acid probe may be modified with.

FIG. 7 depicts the Jump-qPCR strategy. Cell-free DNA or fragmented genomic DNA can be crosslinked with jump-probe that contains a universal sequence, followed by primer extension. The released newly synthesized strands were annealed with designed loci specific primer and subjected to qPCR.

FIG. 8 depicts the Jump-array strategy. Cell free DNA or fragmented genomic DNA can be crosslinked with jump-probe that contains fluorophore, followed by primer extension. The released newly synthesized fluorescent strands were subjected to microarray.

### DETAILED DESCRIPTION OF THE INVENTION

DNA epigenetic modifications such as 5-methylcytosine (5mC) and 5-hydroxymethylcytosine (5hmC) play key roles in biological functions and various diseases. Currently, most common technique for studying cytosine modification is the bisulfite treatment-based sequencing. This technique has major drawbacks in not being able to differentiate 5mC and 5hmC (5-hydroxymethylcytosine), and harsh conditions are required. Readily available and robust technologies for clinical diagnostic of cytosine modifications are very limited. The inventors present a method for identifying 5hmC or 5mC or for distinguishing 5hmC from 5mC in a nucleic acid and specific site detection of 5hmC or 5mC for clinical or other applications in an economic and highly efficient way. In the case of 5hmC detection, this approach involves the following steps: a. modifying endogenous or pre-existing 5hmC in a nucleic acid with a first functional group; b. covalently attaching a modified nucleic acid probe comprising a second functional group to the first functional group; wherein the nucleic acid probe and nucleic acid molecule are covalently linked through the first and second functional groups; c. annealing a primer to the nucleic acid probe; d. performing primer extension of the annealed primer to make a new strand; and e. detecting the new strand.

When 5mC is to be detected, the method first comprises protecting endogenous 5hmC (i.e. with a modification such as a glucose molecule) and converting the endogenous 5mC to 5hmC. For example, this approach involves the following steps: a. modifying 5-hmC nucleic acid bases with a glucose molecule; b. oxidizing 5-mC to 5-hmC to make converted 5-hmC; c. modifying the converted 5-hmC nucleic acid base with a first functional group; d. covalently attaching a modified nucleic acid probe comprising a second functional group to the first functional group; wherein the nucleic acid probe and nucleic acid molecule are covalently linked through the first and second functional groups; e. annealing a primer to the nucleic acid probe; f. performing primer extension of the annealed primer to make a new strand; and g. detecting the new strand.

### I. Nucleotide Modification

A. Oxidation of 5mC for Detection, Sequencing, and Diagnostic Methods

1. Oxidizing 5mC to 5hmC. Oxidation of 5mC to 5hmC can be accomplished by contacting the modified nucleic acid of step 1 with a methylcytosine dioxygenases (e.g., TET1, TET2 and TET3) or an enzyme having similar activity; or chemical modification.

In some embodiments, it is contemplated that TET1, TET2, or TET3 are human or mouse proteins. Human TET1 has accession number NM_030625.2; human TET2 has accession number NM_001127208.2, alternatively, NM_017628.4; and human TET3 has accession number NM_144993.1. Mouse TET1 has accession number NM_027384.1; mouse TET2 has accession number NM_001040400.2; and mouse TET3 has accession number NM_183138.2.

B. Modification of 5hmC

Certain embodiments are directed to methods and compositions for modifying 5hmC, detecting 5hmC, and/or evaluating 5hmC in nucleic acids. In certain aspects, 5hmC is glycosylated. In a further aspect 5hmC is coupled to a modified, unmodified, and/or labeled glucose moiety. In certain aspects a target nucleic acid is contacted with a β-glucosyltransferase enzyme and a UDP substrate comprising an unmodified, modified, or modifiable glucose moiety. Using the methods described herein a large variety of detectable groups (biotin, fluorescent tag, radioactive groups, etc.) can be coupled to 5hmC via a glucose modification. Methods and compositions are described in PCT application PCT/US2011/031370, filed Apr. 6, 2011, which is hereby incorporated by reference in its entirety.

The methods described herein relate to covalently attaching a modified nucleic acid probe to 5hmC via the glucose modification.

Modification of 5hmC can be performed using the enzyme β-glucosyltransferase (βGT), or a similar enzyme, that catalyzes the transfer of a glucose moiety from uridine diphosphoglucose (UDP-Glc) to the hydroxyl group of 5hmC, yielding β-glycosyl-5-hydroxymethyl-cytosine (5gmC). The inventors have found that this enzymatic glycosylation offers a strategy for incorporating modified glucose molecules for labeling or tagging 5hmC in eukaryotic nucleic acids. For instance, a glucose molecule chemically modified to contain an azide (N₃) group may be covalently attached to 5hmC through this enzyme-catalyzed glycosylation. Thereafter, the modified nucleic acid probe can be specifically installed onto glycosylated 5hmC via reactions with the azide.

The inventors have shown that a functional group (e.g., an azide group) can be incorporated into DNA using methods described herein. This incorporation of a functional group allows further labeling or tagging cytosine residues with a nucleic acid probe and other tags. The labeling or tagging of 5hmC can use, for example, click chemistry or other functional/coupling groups know to those skilled in the art. The labeled or tagged DNA fragments containing 5hmC can be isolated and/or evaluated using the methods of the disclosure.

C. TET Proteins

The ten-eleven translocation (TET) proteins are a family of DNA hydroxylases that have been discovered to have enzymatic activity toward the methyl group on the 5-position of cytosine (5-methylcytosine [5mC]). The TET protein family includes three members, TET1, TET2, and TET3. TET proteins are believed to have the capacity of converting 5mC into 5-hydroxymethylcytosine (5hmC), 5-formylcytosine (5fC), and 5-carboxylcytosine (5caC) through three consecutive oxidation reactions.

The first member of TET family proteins, TET1 gene, was first detected in acute myeloid leukemia (AML) as a fusion partner of the histone H3 Lys 4 (H3K4)methyltransferase MLL (mixed-lineage leukemia) (Ono et al., 2002; Lorsbach et al., 2003). It has been first discovered that human TET1 protein possesses enzymatic activity capable of hydroxylating 5mC to generate 5hmC (Tahiliani et al., 2009). Later on, all members of the mouse TET protein family (TET 1-3) have been demonstrated to have 5mC hydroxylase activities (Ito et al., 2010).

TET proteins generally possess several conserved domains, including a CXXC zinc finger domain which has high affinity for clustered unmethylated CpG dinucleotides, a catalytic domain that is typical of Fe(II)- and 2-oxoglutarate (20G)-dependent dioxygenases, and a cysteine-rich region (Wu and Zhang, 2011, Tahiliani et al., 2009).

D. β-glycosyltransferase (β-GT)

A glucosyl-DNA beta-glucosyltransferase (EC 2.4.1.28, β-glycosyltransferase (βGT)) is an enzyme that catalyzes the chemical reaction in which a beta-D-glucosyl residue is transferred from UDP-glucose to a glucosylhydroxymethylcytosine residue in a nucleic acid. This enzyme resembles DNA beta-glucosyltransferase in that respect. This enzyme belongs to the family of glycosyltransferases, specifically the hexosyltransferases. The systematic name of this enzyme class is UDP-glucose:D-glucosyl-DNA beta-D-glucosyltransferase. Other names in common use include T6-glucosyl-HMC-beta-glucosyl transferase, T6-beta-glucosyl transferase, uridine diphosphoglucose-glucosyldeoxyribonucleate, and beta-glucosyltransferase.

In certain aspects, the a β-glucosyltransferase is a His-tag fusion protein having the amino acid sequence (βGT begins at amino acid 25(met)):

(SEQ ID NO: 1)
SHHHHHHSSGVDLGTENLYFQSNAMKIAIINMGNNVINFKTVPSSETIYL
FKVISEMGLNVDIISLKNGVYTKSFDEVDVNDYDRLIVVNSSINFFGGKP
NLAILSAQKFMAKYKSKIYYLFTDIRLPFSQSWPNVKNRPWAYLYTEEEL
LIKSPIKVISQGINLDIAKAAHKKVDNVIEFEYFPIEQYKIHMNDFQLSK
PTKKTLDVIYGGSFRSGQRESKMVEFLFDTGLNIEFFGNAREKQFKNPKY
PWTKAPVFTGKIPMNMVSEKNSQAIAALIIGDKNYNDNFITLRVWETMAS
DAVMLIDEEFDTKHRIINDARFYVNNRAELIDRVNELKHSDVLRKEMLSI
QHDILNKTRAKKAEWQDAFKKAIDL.

In other embodiments, the protein may be used without the His-tag (hexa-histidine tag shown above) portion. For example, βGT was cloned into the target vector pMCSG19 by Ligation Independent Cloning (LIC) method according to Donnelly et al. (2006). The resulting plasmid was transformed into BL21 star (DE3) competent cells containing pRK1037 (Science Reagents, Inc.) by heat shock. Positive colonies were selected with 150 μg/ml Ampicillin and 30 μg/ml Kanamycin. One liter of cells was grown at 37° C. from a 1:100 dilution of an overnight culture. The cells were induced with 1 mM of IPTG when OD600 reaches 0.6-0.8. After overnight growth at 16° C. with shaking, the cells were collected by centrifugation, suspended in 30 mL Ni-NTA buffer A (20 mM Tris-HCl pH 7.5, 150 mM NaCl, 30 mM imidazole, and 10 mM β-ME) with protease inhibitor PMSF. After loading to a Ni-NTA column, proteins were eluted with a 0-100% gradient of Ni-NTA buffer B (20 mM Tris-HCl pH 7.5, 150 mM NaCl, 400 mM imidazole, and 10 mM β-ME). βGT-containing fractions were further purified by MonoS (Buffer A: 10 mM Tris-HCl pH 7.5; Buffer B: 10 mM Tris-HCl pH 7.5, and 1M NaCl) to remove DNA. Finally, the collected protein fractions were loaded onto a Superdex 200 (GE) gel-filtration column equilibrated with 50 mM Tris-HCl pH 7.5, 20 mM MgCl₂, and 10 mM SDS-PAGE gel revealed a high degree of purity of βGT. βGT was concentrated to 45 μM and stored frozen at −80° C. with an addition of 30% glycerol.

A variety of proteins can be purified using methods known in the art. Protein purification is a series of processes intended to isolate a single type of protein from a complex mixture. Protein purification is vital for the characterization of the function, structure and interactions of the protein of interest. The starting material is usually a biological tissue or a microbial culture. The various steps in the purification process may free the protein from a matrix that confines it, separate the protein and non-protein parts of the mixture, and finally separate the desired protein from all other proteins. Separation of one protein from all others is typically the most laborious aspect of protein purification. Separation steps exploit differences in protein size, physico-chemical properties and binding affinity.

Evaluating Purification Yield.

The most general method to monitor the purification process is by running a SDS-PAGE of the different steps. This method only gives a rough measure of the amounts of different proteins in the mixture, and it is not able to distinguish between proteins with similar molecular weight. If the protein has a distinguishing spectroscopic feature or an enzymatic activity, this property can be used to detect and quantify the specific protein, and thus to select the fractions of the separation, that contains the protein. If antibodies against the protein are available then western blotting and ELISA can specifically detect and quantify the amount of desired protein. Some proteins function as receptors and can be detected during purification steps by a ligand binding assay, often using a radioactive ligand.

In order to evaluate the process of multistep purification, the amount of the specific protein has to be compared to the amount of total protein. The latter can be determined by the Bradford total protein assay or by absorbance of light at 280 nm, however some reagents used during the purification process may interfere with the quantification. For example, imidazole (commonly used for purification of polyhistidine-tagged recombinant proteins) is an amino acid analogue and at low concentrations will interfere with the bicinchoninic acid (BCA) assay for total protein quantification. Impurities in low-grade imidazole will also absorb at 280 nm, resulting in an inaccurate reading of protein concentration from UV absorbance.

Another method to be considered is Surface Plasmon Resonance (SPR). SPR can detect binding of label free molecules on the surface of a chip. If the desired protein is an antibody, binding can be translated to directly to the activity of the protein. One can express the active concentration of the protein as the percent of the total protein. SPR can be a powerful method for quickly determining protein activity and overall yield. It is a powerful technology that requires an instrument to perform.

Methods of Protein Purification.

The methods used in protein purification can roughly be divided into analytical and preparative methods. The distinction is not exact, but the deciding factor is the amount of protein that can practically be purified with that method. Analytical methods aim to detect and identify a protein in a mixture, whereas preparative methods aim to produce large quantities of the protein for other purposes, such as structural biology or industrial use.

Depending on the source, the protein has to be brought into solution by breaking the tissue or cells containing it. There are several methods to achieve this: Repeated freezing and thawing, sonication, homogenization by high pressure, filtration (either via cellulose-based depth filters or cross-flow filtration), or permeabilization by organic solvents. The method of choice depends on how fragile the protein is and how sturdy the cells are. After this extraction process soluble proteins will be in the solvent, and can be separated from cell membranes, DNA etc. by centrifugation. The extraction process also extracts proteases, which will start digesting the proteins in the solution. If the protein is sensitive to proteolysis, it is usually desirable to proceed quickly, and keep the extract cooled, to slow down proteolysis.

In bulk protein purification, a common first step to isolate proteins is precipitation with ammonium sulfate (NH₄)₂SO₄. This is performed by adding increasing amounts of ammonium sulfate and collecting the different fractions of precipitate protein. One advantage of this method is that it can be performed inexpensively with very large volumes.

The first proteins to be purified are water-soluble proteins. Purification of integral membrane proteins requires disruption of the cell membrane in order to isolate any one particular protein from others that are in the same membrane compartment. Sometimes a particular membrane fraction can be isolated first, such as isolating mitochondria from cells before purifying a protein located in a mitochondrial membrane. A detergent such as sodium dodecyl sulfate (SDS) can be used to dissolve cell membranes and keep membrane proteins in solution during purification; however, because SDS causes denaturation, milder detergents such as Triton X-100 or CHAPS can be used to retain the protein's native conformation during complete purification.

Centrifugation is a process that uses centrifugal force to separate mixtures of particles of varying masses or densities suspended in a liquid. When a vessel (typically a tube or bottle) containing a mixture of proteins or other particulate matter, such as bacterial cells, is rotated at high speeds, the angular momentum yields an outward force to each particle that is proportional to its mass. The tendency of a given particle to move through the liquid because of this force is offset by the resistance the liquid exerts on the particle. The net effect of “spinning” the sample in a centrifuge is that massive, small, and dense particles move outward faster than less massive particles or particles with more “drag” in the liquid. When suspensions of particles are “spun” in a centrifuge, a “pellet” may form at the bottom of the vessel that is enriched for the most massive particles with low drag in the liquid. Non-compacted particles still remaining mostly in the liquid are called the “supernatant” and can be removed from the vessel to separate the supernatant from the pellet. The rate of centrifugation is specified by the angular acceleration applied to the sample, typically measured in comparison to the g. If samples are centrifuged long enough, the particles in the vessel will reach equilibrium wherein the particles accumulate specifically at a point in the vessel where their buoyant density is balanced with centrifugal force. Such an “equilibrium” centrifugation can allow extensive purification of a given particle.

Sucrose gradient centrifugation is a linear concentration gradient of sugar (typically sucrose, glycerol, or a silica based density gradient media, like Percoll™) is generated in a tube such that the highest concentration is on the bottom and lowest on top. A protein sample is then layered on top of the gradient and spun at high speeds in an ultracentrifuge. This causes heavy macromolecules to migrate towards the bottom of the tube faster than lighter material. After separating the protein/particles, the gradient is then fractionated and collected.

Usually a protein purification protocol contains one or more chromatographic steps. The basic procedure in chromatography is to flow the solution containing the protein through a column packed with various materials. Different proteins interact differently with the column material, and can thus be separated by the time required to pass the column, or the conditions required to elute the protein from the column. Usually proteins are detected as they are coming off the column by their absorbance at 280 nm. Many different chromatographic methods exist.

Chromatography can be used to separate protein in solution or denaturing conditions by using porous gels. This technique is known as size exclusion chromatography. The principle is that smaller molecules have to traverse a larger volume in a porous matrix. Consequentially, proteins of a certain range in size will require a variable volume of eluent (solvent) before being collected at the other end of the column of gel.

In the context of protein purification, the eluant is usually pooled in different test tubes. All test tubes containing no measurable trace of the protein to purify are discarded. The remaining solution is thus made of the protein to purify and any other similarly-sized proteins.

Ion exchange chromatography separates compounds according to the nature and degree of their ionic charge. The column to be used is selected according to its type and strength of charge. Anion exchange resins have a positive charge and are used to retain and separate negatively charged compounds, while cation exchange resins have a negative charge and are used to separate positively charged molecules. Before the separation begins a buffer is pumped through the column to equilibrate the opposing charged ions. Upon injection of the sample, solute molecules will exchange with the buffer ions as each competes for the binding sites on the resin. The length of retention for each solute depends upon the strength of its charge. The most weakly charged compounds will elute first, followed by those with successively stronger charges. Because of the nature of the separating mechanism, pH, buffer type, buffer concentration, and temperature all play important roles in controlling the separation.

Affinity Chromatography is a separation technique based upon molecular conformation, which frequently utilizes application specific resins. These resins have ligands attached to their surfaces which are specific for the compounds to be separated. Most frequently, these ligands function in a fashion similar to that of antibody-antigen interactions. This “lock and key” fit between the ligand and its target compound makes it highly specific, frequently generating a single peak, while all else in the sample is unretained.

Many membrane proteins are glycoproteins and can be purified by lectin affinity chromatography. Detergent-solubilized proteins can be allowed to bind to a chromatography resin that has been modified to have a covalently attached lectin. Proteins that do not bind to the lectin are washed away and then specifically bound glycoproteins can be eluted by adding a high concentration of a sugar that competes with the bound glycoproteins at the lectin binding site. Some lectins have high affinity binding to oligosaccharides of glycoproteins that is hard to compete with sugars, and bound glycoproteins need to be released by denaturing the lectin.

A common technique involves engineering a sequence of 6 to 8 histidines into the N- or C-terminal of the protein. The polyhistidine binds strongly to divalent metal ions such as nickel and cobalt. The protein can be passed through a column containing immobilized nickel ions, which binds the polyhistidine tag. All untagged proteins pass through the column. The protein can be eluted with imidazole, which competes with the polyhistidine tag for binding to the column, or by a decrease in pH (typically to 4.5), which decreases the affinity of the tag for the resin. While this procedure is generally used for the purification of recombinant proteins with an engineered affinity tag (such as a 6×His tag or Clontech's HAT tag), it can also be used for natural proteins with an inherent affinity for divalent cations.

Immunoaffinity chromatography uses the specific binding of an antibody to the target protein to selectively purify the protein. The procedure involves immobilizing an antibody to a column material, which then selectively binds the protein, while everything else flows through. The protein can be eluted by changing the pH or the salinity. Because this method does not involve engineering in a tag, it can be used for proteins from natural sources.

Another way to tag proteins is to engineer an antigen peptide tag onto the protein, and then purify the protein on a column or by incubating with a loose resin that is coated with an immobilized antibody. This particular procedure is known as immunoprecipitation. Immunoprecipitation is quite capable of generating an extremely specific interaction which usually results in binding only the desired protein. The purified tagged proteins can then easily be separated from the other proteins in solution and later eluted back into clean solution. Tags can be cleaved by use of a protease. This often involves engineering a protease cleavage site between the tag and the protein.

High performance liquid chromatography or high pressure liquid chromatography is a form of chromatography applying high pressure to drive the solutes through the column faster. This means that the diffusion is limited and the resolution is improved. The most common form is “reversed phase” hplc, where the column material is hydrophobic. The proteins are eluted by a gradient of increasing amounts of an organic solvent, such as acetonitrile. The proteins elute according to their hydrophobicity. After purification by HPLC the protein is in a solution that only contains volatile compounds, and can easily be lyophilized. HPLC purification frequently results in denaturation of the purified proteins and is thus not applicable to proteins that do not spontaneously refold.

At the end of a protein purification, the protein often has to be concentrated. Different methods exist. If the solution doesn't contain any other soluble component than the protein in question the protein can be lyophilized (dried). This is commonly done after an HPLC run. This simply removes all volatile component leaving the proteins behind.

Ultrafiltration concentrates a protein solution using selective permeable membranes. The function of the membrane is to let the water and small molecules pass through while retaining the protein. The solution is forced against the membrane by mechanical pump or gas pressure or centrifugation.

Gel electrophoresis is a common laboratory technique that can be used both as preparative and analytical method. The principle of electrophoresis relies on the movement of a charged ion in an electric field. In practice, the proteins are denatured in a solution containing a detergent (SDS). In these conditions, the proteins are unfolded and coated with negatively charged detergent molecules. The proteins in SDS-PAGE are separated on the sole basis of their size.

In analytical methods, the protein migrate as bands based on size. Each band can be detected using stains such as Coomassie blue dye or silver stain. Preparative methods to purify large amounts of protein, require the extraction of the protein from the electrophoretic gel. This extraction may involve excision of the gel containing a band, or eluting the band directly off the gel as it runs off the end of the gel.

In the context of a purification strategy, denaturing condition electrophoresis provides an improved resolution over size exclusion chromatography, but does not scale to large quantity of proteins in a sample as well as the late chromatography columns.

E. Modification Moieties

5mC and/or 5hmC can be directly or indirectly modified with a number of functional groups or labeled molecules. One example is the oxidation of 5mC and the subsequent labeling with a functionalized, protectant, or labeled glucose molecule. In certain embodiments, 5mC can be first modified with a modification moiety or a functional group prior to being further modified by the attachment of a glucosyl moiety.

In additional embodiments, a functionalized or labeled glucose molecule can be used in conjunction with βGT to modify 5hmC in a nucleic polymer such as DNA or RNA. In certain aspects, the βGT UDP substrate comprises a functionalized or labeled glucose moiety.

In a further aspect, the modification moiety can be modified or functionalized using click chemistry or other coupling chemistries known in the art. Click chemistry is a chemical philosophy introduced by K. Barry Sharpless in 2001 (Kolb et al., 2001; Evans, 2007) and describes chemistry tailored to generate substances quickly and reliably by joining small units.

1. Functional Groups

Chemical reactions that lead to a covalent linkage include, for example, cycloaddition reactions (such as the Diels-Alder's reaction, the 1,3-dipolar cycloaddition Huisgen reaction, and the similar “click reaction”), condensations, nucleophilic and electrophilic addition reactions, nucleophilic and electrophilic substitutions, addition and elimination reactions, alkylation reactions, rearrangement reactions and any other known organic reactions that involve a functional group.

Representative examples of functional groups include, without limitation, acyl halide, aldehyde, alkoxy, alkyne, amide, amine, aryloxy, azide, aziridine, azo, carbamate, carbonyl, carboxyl, carboxylate, cyano, diene, dienophile, epoxy, guanidine, guanyl, halide, hydrazide, hydrazine, hydroxy, hydroxylamine, imino, isocyanate, nitro, phosphate, phosphonate, sulfinyl, sulfonamide, sulfonate, thioalkoxy, thioaryloxy, thiocarbamate, thiocarbonyl, thiohydroxy, thiourea and urea, as these terms are defined hereinafter.

Exemplary first and second functional groups that are chemically compatible with one another as described herein include, but are not limited to, hydroxy and carboxylic acid, which form an ester bond; thiol and carboxylic acid, which form a thioester bond; amine and carboxylic acid, which form an amide bond; aldehyde and amine, hydrazine, hydrazide, hydroxylamine, phenylhydrazine, semicarbazide or thiosemicarbazide, which form a Schiff base (imine bond); alkene and diene, which react therebetween via cycloaddition reactions; and functional groups that can participate in a Click reaction.

Further examples of pairs of functional groups (first and second functional groups) capable of reacting with one another include an azide and an alkyne, an unsaturated carbon-carbon bond (e.g., acrylate, methacrylate, maleimide) and a thiol, an unsaturated carbon-carbon bond and an amine, a carboxylic acid and an amine, a hydroxyl and an isocyanate, a carboxylic acid and an isocyanate, an amine and an isocyanate, a thiol and an isocyanate. Additional examples include an amine, a hydroxyl, a thiol or a carboxylic acid along with a nucleophilic leaving group (e.g., hydroxysuccinimide, a halogen).

It is to be appreciated that for each pair of functional groups described hereinabove, either functional group can correspond to the “first functional group” or to the “second functional group”.

In some embodiments, the first and/or the second functional groups can be latent groups, which are exposed during the chemical reaction, such that the reacting (e.g., covalent bond formation) is effected once a latent group is exposed. Exemplary such groups include, but are not limited to, functional groups as described hereinabove, which are protected with a protecting group that is labile under selected reaction conditions.

Examples of labile protecting groups include, for example, carboxylate esters, which may hydrolyzed to form an alcohol and a carboxylic acid by exposure to acidic or basic conditions; silyl ethers such as trialkyl silyl ethers, which can be hydrolysed to an alcohol by acid or fluoride ion; p-methoxybenzyl ethers, which may be hydrolysed to an alcohol, for example, by oxidizing conditions or acidic conditions; t-butyloxycarbonyl and 9-fluorenylmethyloxycarbonyl, which may be hydrolysed to an amine by a exposure to basic conditions; sulfonamides, which may be hydrolysed to a sulfonate and amine by exposure to a suitable reagent such as samarium iodide or tributyltin hydride; acetals and ketals, which may be hydrolysed to form an aldehyde or ketone, respectively, along with an alcohol or diol, by exposure o acidic conditions; acylals (i.e., wherein a carbon atom is attached to two carboxylate groups), which may be hydrolysed to an aldehyde of ketone, for example, by exposure to a Lewis acid; orthoesters (i.e., wherein a carbon atom is attached to three alkoxy or aryloxy groups), which may be hydrolysed to a carboxylate ester (which may be further hydrolysed as described hereinabove) by exposure to mildly acidic conditions; 2-cyanoethyl phosphates, which may be converted to a phosphate by exposure to mildly basic conditions; methylphosphates, which may be hydrolysed to phosphates by exposure to strong nucleophiles; phosphates, which may be hydrolysed to alcohols, for example, by exposure to phosphatases; and aldehydes, which may be converted to carboxylic acids, for example, by exposure to an oxidizing agent.

According to some embodiments of the current disclosure, a linking moiety is formed as a result of a bond-forming reaction between two (first and second) functional groups.

Exemplary linking moieties, according to some embodiments of the present invention, which are formed between a first and a second functional groups as described herein include without limitation, amide, lactone, lactam, carboxylate (ester), cycloalkene (e.g., cyclohexene), heteroalicyclic, heteroaryl, triazine, triazole, disulfide, imine, aldimine, ketimine, hydrazone, semicarbazone and the likes. Other linking moieties are defined hereinbelow.

For example, a reaction between a diene functional group and a dienophile functional group, e.g. a Diels-Alder reaction, would form a cycloalkene linking moiety, and in most cases a cyclohexene linking moiety. In another example, an amine functional group would form an amide linking moiety when reacted with a carboxyl functional group. In another example, a hydroxyl functional group would form an ester linking moiety when reacted with a carboxyl functional group. In another example, a sulfhydryl functional group would form a disulfide (—S—S—) linking moiety when reacted with another sulfhydryl functional group under oxidation conditions, or a thioether (thioalkoxy) linking moiety when reacted with a halo functional group or another leaving-functional group. In another example, an alkynyl functional group would form a triazole linking moiety by “click reaction” when reacted with an azide functional group.

The “click reaction”, also known as “click chemistry” is a name often used to describe a stepwise variant of the Huisgen 1,3-dipolar cycloaddition of azides and alkynes to yield 1,2,3-triazole. This reaction is carried out under ambient conditions, or under mild microwave irradiation, typically in the presence of a Cu(I) catalyst, and with exclusive regioselectivity for the 1,4-disubstituted triazole product when mediated by catalytic amounts of Cu(I) salts [V. Rostovtsev, L. G. Green, V. V. Fokin, K. B. Sharpless, Angew. Chem. Int. Ed. 2002, 41, 2596; H. C. Kolb, M. Finn, K. B. Sharpless, Angew Chem., Int. Ed. 2001, 40, 2004].

The “click reaction” is particularly suitable in the context of embodiments of the present invention since it can be carried out under conditions which are non-distructive to DNA molecules, and it affords attachment of a labeling agent to 5hmC in a DNA molecule at high chemical yields using mild conditions in aqueous media. The selectivity of this reaction allows to perform the reaction with minimized or nullified use of protecting groups, which use often results in multistep cumbersome synthetic processes.

In exemplary embodiments, the first and second functional groups comprise (in no particular order) an azide and an alkyne. These two functional groups may combine to form a triazole ring, as a linking moiety. These two functional groups thus combine to attach a nucleic acid probe to the 5hmC in the DNA molecule by a mechanism referred to as “click” chemistry.

The functional groups may be convalently attached to and/or further comprise a molecule such as a glucose or modified glucose or a sterically bulky molecule. In some embodiments, a modified glucose molecule comprising a functional group is covalently attached to the 5hmC to make a 5gmC. In this embodiment, one of the hydroxy groups of a glucose can be substituted by a chemical moiety that comprises the first functional group or can be used to attach to the glucose the chemical moiety that comprises the first functional group, via chemical reactions that involve a hydroxy group, as described herein.

In exemplary embodiments, one of the hydroxy groups of a glucose is substituted (replaced) by a chemical moiety that comprises the first functional group. Chemical reactions for substituting a hydroxy group are well known in the art.

In some embodiments, the first functional group is azide and a hydroxy at position 6 of the glucose is substituted by an azide group.

In some embodiments of the disclosure, a DNA molecule in which the 5-hydroxymethylcytosine bases are glycosylated by a glucose molecule modified with the first functional group is prepared.

In some embodiments, a selective introduction of a glucose modified with the first functional group to 5-hydroxymethylcytosines in a DNA molecule comprises incubating the DNA molecule with β-glucosyltransferase and a uridine diphosphoglucose (UDP-Glu) modified with the first functional group.

As discussed herein, in some embodiments, the reaction involves a click chemistry reaction.

A uridine diphosphoglucose (UDP-Glu) modified with the first functional group is meant to describe a uridine diphosphoglucose in which the glucose moiety is derivatized by a first functional group. In some embodiments, the uridine diphosphoglucose (UDP-Glu) modified with the first functional group is a UDP-6-N₃-Glucose.

A UDP-6-N₃-Glucose, or any other uridine diphosphoglucose (UDP-Glu) modified with the first functional group, can be prepared by chemical synthesis, while utilizing, for example, a 6-azido glucose or any other derivatized glucose, or can be a commercially available product.

In some embodiments, the UDP-6-N.sub.3-Glucose, or any other uridine diphosphoglucose (UDP-Glu) derivatized by the first reactive group, is prepared by enzymatically-catalyzed reactions, as exemplified in further detail hereinafter.

Once a glucose modified with a first functional group is introduced to 5hmCs in a DNA molecule, the DNA molecule is reacted with a nucleic acid probe comprising a compatible second functional group, as described herein.

According to some embodiments of the invention, the click chemistry reaction is free of a copper catalyst, namely, is effected without the presence of a copper catalyst or any other catalyst that may adversely affect the DNA molecule.

2. Transposone Labeling of DNA

In certain aspects the nucleic acid molecule is tagged with a transposon. For example, the nucleic acid molecule may be contacted with a transposon and a transposase to allow for the non-specific integration of the transposon into the nucleic acid molecule.

As used throughout, the term transposon refers to a double-stranded DNA that contains the nucleotide sequences that are necessary to form the complex with the transposase or integrase enzyme that is functional in an in vitro transposition reaction. A transposon forms a complex or a synaptic complex or a transposome complex. The transposon can also form a transposome composition with a transposase or integrase that recognizes and binds to the transposon sequence, and which complex is capable of inserting or transposing the transposon into target DNA with which it is incubated in an in vitro transposition reaction.

Tagging the nucleic acid molecule with a transposon may also include fragmenting the tagged DNA. In some embodiments, a transposase may be used to catalyze integration of oligonucleotides into a target nucleic acid at high density (e.g. at about every 300 base pairs). For example, a transposase, such as Nextera's TRANSPOSOME™ technology, may be used to generate random dsDNA breaks. The TRANSPOSOME™ complex includes free transposon ends and a transposase. When this complex is incubated with dsDNA, the DNA is fragmented and the transferred strand of the transposon end oligonucleotide is covalently attached to the end of the DNA fragment. In some embodiments, it is attached to the 3′ end. In some embodiments, it is attached to the 5′ end. In some applications, the transposon ends may be appended with primer sites. By varying buffer and reaction conditions (e.g., concentration of TRANSPOSOME™ complexes), the size distribution of the fragmented and tagged DNA library may be controlled. In some embodiments, the transposon comprises a P7 adapter having the following sequence: GTCTCGTGGGCTCGGAGATGTGTATAAGAGACAG (SEQ ID NO:2). In some embodiments, the transposase comprises Tn5 and/or a derivative thereof. Derivatives of Tn5 are known in the art and commercially available.

In some embodiments, the transposon further comprises a label or affinity tag, such as biotin. Other affinity tags include E-tag, Flag-tag, HA-tag, His-tag, Myc-tag, etc. In some embodiments, the affinity tag is attached to the end of the P7 adapter. In some embodiments, the affinity tag is attached to the 5′ end of the adapter.

3. Synthesis of Modified Uridine Diphosphate Glucose (UDP-Glu) Bearing Thiol or Azide.

The initial success of 5hmC glycosylation led to the hypothesis that thiol- or azide-modified glucose can be similarly transferred to 5hmC in duplex DNA. Thus, the inventors have synthesized azide-substituted UDP-Glu and contemplate synthesizing thiol-substituted UDP-Glu for 5hmC labeling. An azide tag is one specific embodiment because this functional group is not present inside cells. The click chemistry to label this group is completely bio-orthogonal, meaning no interference from biological samples (Kolb et al., 2001). The azide-substituted glucoses can be transferred to 5hmC, see Song et al., 2011, which is incorporated herein by reference.

4. Nucleic Acid Probes

In methods of the disclosure, a nucleic acid probe is covalently attached to a nucleic acid. This nucleic acid probe facilitates attachment of a primer that, once a polymerase is added, can allow for primer extension and new strand synthesis at the site of attachment of the nucleic acid probe. Subsequent sequencing of the new strand can reveal the location of modified cytosines. In some embodiments, the nucleic acid probe is a DNA probe. In some embodiments, the nucleic acid probe is an RNA probe. The nucleic acid probe is covalently attached to the nucleic acid by the functional group on the nucleic acid probe.

The sequence of the nucleic acid probe is a known sequence, which allows for the construction of a primer that is capable of annealing to the probe and facilitating primer extension and new strand synthesis. In some embodiments, the primer is covalently attached to the nucleic acid probe. Therefore, the primer may be a nucleic acid sequence that is contiguous with the nucleic acid probe. In some embodiments, the primer comprises a P5 adapter sequence: CGTCGGCAGCGTC (SEQ ID NO:3). In some embodiments, the nucleic acid probe comprises the following sequence: CGAGTCANNNNNNNNCTGTCTCTTATACACATCTGACGCTGCCGdUdUdUTCGTC GGCAGCGTC (SEQ ID NO:4), wherein N is any nucleic acid base.

In some embodiments, the nucleic acid probe comprises a hairpin. In some embodiments, the hairpin comprises a loop region, wherein the loop region is cleavable to allow for the release of the new strand after new strand synthesis. In some embodiments, the loop region comprises deoxyribose uracils, which allows for the cleavage of the loop region with a uracil DNA glycosylase, such as a USER™ enzyme.

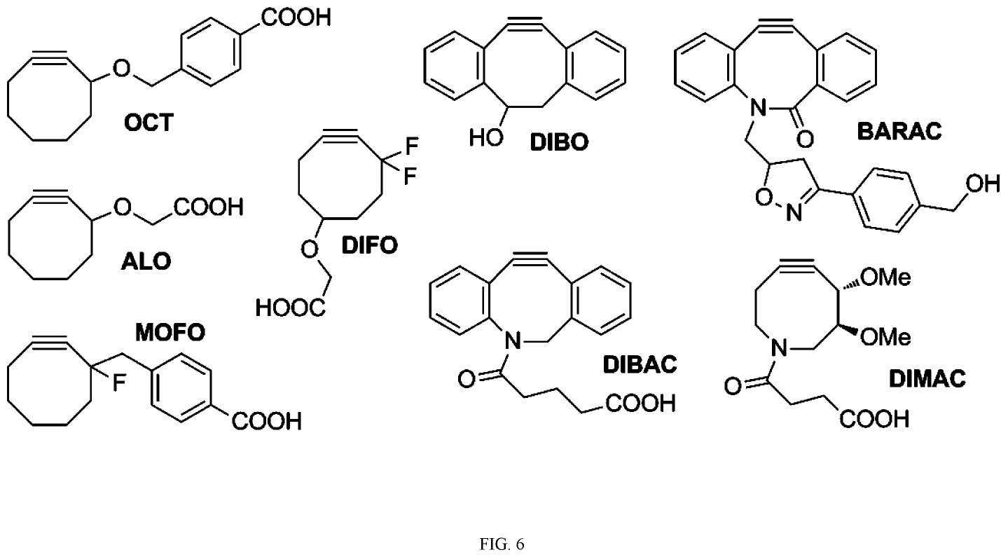

In the methods described herein, the nucleic acid probe may be modified with a molecule that has a molecular mass or weight of at least 75, 100, 110, 115, 120, 125, 130, 135, 140, 145, 150, 160, 170, 180, 190, 200, 210, 220, 230, 240, 250, 260, 270, 280, 290 or 300, or any derivable range therein. In some embodiments, the molecule is a cyclooctyne derivative. Exemplary molecules that the nucleic acid probe may be modified with include DBCO (Dibenzocyclooctyl), polyethylene glycol polymers, and those molecules shown in FIG. 6.

### II. Sequencing Methods

A. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

B. Polony sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

C. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

D. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massively parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

E. Solid Sequencing.

Applied Biosystems' (now a Life Technologies brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

F. Ion Torrent Semiconductor Sequencing.

Ion Torrent Systems Inc. (now owned by Life Technologies) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

G. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects and is scheduled to be used for more.

H. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

I. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

### III. Labels

The oligonucleotides, nucleic acids, primers, and/or probes of the disclosure may include one or more labels. Nucleic acid molecules can be labeled by incorporating moieties detectable by one or more means including, but not limited to, spectroscopic, photochemical, biochemical, immunochemical, or chemical assays. The method of linking or conjugating the label to the nucleotide or oligonucleotide depends on the type of label(s) used and the position of the label on the nucleotide or oligonucleotide.

As used herein, “labels” are chemical or biochemical moieties useful for labeling a nucleic acid. “Labels” include, for example, fluorescent agents, chemiluminescent agents, chromogenic agents, quenching agents, radionucleotides, enzymes, substrates, cofactors, inhibitors, nanoparticles, magnetic particles, and other moieties known in the art. Labels are capable of generating a measurable signal and may be covalently or noncovalently joined to an oligonucleotide or nucleotide.

In some embodiments, the nucleic acid molecules may be labeled with a “fluorescent dye” or a “fluorophore.” As used herein, a “fluorescent dye” or a “fluorophore” is a chemical group that can be excited by light to emit fluorescence. Some fluorophores may be excited by light to emit phosphorescence. Dyes may include acceptor dyes that are capable of quenching a fluorescent signal from a fluorescent donor dye. Dyes that may be used in the disclosed methods include, but are not limited to, the following dyes sold under the following trade names: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-Hydroxy Tryptamine (HAT); 5-ROX (carboxy-X-rhodamine); 6-Carboxyrhodamine 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Cascade Blue™; Cascade Yellow; Catecholamine; CCF2 (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NETS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant Iavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PYMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The nucleic acid molecules of the disclosed compositions and methods may be labeled with a quencher. Quenching may include dynamic quenching (e.g., by FRET), static quenching, or both. Illustrative quenchers may include Dabcyl. Illustrative quenchers may also include dark quenchers, which may include black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.). Dark quenchers also may include DNP-type non-fluorophores that include a 2,4-dinitrophenyl group.

The labels can be conjugated to the nucleic acid molecules directly or indirectly by a variety of techniques. Depending upon the precise type of label used, the label can be located at the 5′ or 3′ end of the oligonucleotide, located internally in the oligonucleotide's nucleotide sequence, or attached to spacer arms extending from the oligonucleotide and having various sizes and compositions to facilitate signal interactions. Using commercially available phosphoramidite reagents, one can produce nucleic acid molecules containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite. In embodiments in which the probe comprises a cleavage site, the label may be located upstream, downstream, 5′ or 3′ to the cleavage site. In specific embodiments, the label is incorporated into the new strand.

### IV. Kits

The invention additionally provides kits for modifying cytosine bases of nucleic acids and/or subjecting such modified nucleic acids to further analysis. The contents of a kit can include one or more of the following reagents described throughout the disclosure such as modification reagents comprising a first functional group, modified nucleic acid probes described herein, primers, reagents for performing primer extension, such as a polymerase, buffers, and nucleotides, sequencing reagents, sequencing primers, a β-glucosyltransferase, transposome reagents, affinity tags, and/or antibodies that bind to affinity tags.

Each kit may include a 5mC or 5hmC modifying agent or agents, e.g., TET, βGT, modification moiety, etc. One or more reagent is preferably supplied in a solid form or liquid buffer that is suitable for inventory storage, and later for addition into the reaction medium when the method of using the reagent is performed. Suitable packaging is provided. The kit may optionally provide additional components that are useful in the procedure. These optional components include buffers, capture reagents, developing reagents, labels, reacting surfaces, means for detection, control samples, instructions, and interpretive information.

Each kit may also include additional components that are useful for amplifying the nucleic acid, or sequencing the nucleic acid, or other applications of the present disclosure as described herein. The kit may optionally provide additional components that are useful in the procedure. These optional components include buffers, capture reagents, developing reagents, labels, reacting surfaces, means for detection, control samples, instructions, and interpretive information.

### V. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of certain embodiments, are provided as an example, and are not intended as limitations on the scope of the invention. Changes therein and other uses which are encompassed within the spirit of the invention as defined by the scope of the claims will occur to those skilled in the art.

Nucleic acid analysis and evaluation includes various methods of amplifying, fragmenting, and/or hybridizing nucleic acids that have or have not been modified.

A. Genomic Analysis

Methodologies are available for large scale sequence analysis. In certain aspects, the methods described exploit these genomic analysis methodologies and adapt them for uses incorporating the methodologies described herein. In certain instances the methods can be used to perform high resolution methylation and/or hydroxymethylation analysis on several thousand CpGs in genomic DNA. Therefore, methods are directed to analysis of the methylation and/or hydroxymethylation status of a genomic DNA sample.

The present methods allow for analyzing the methylation and/or hydroxymethylation status of all regions of a complete genome, where changes in methylation and/or hydroxymethylation status are expected to have an influence on gene expression. Due to the combination of the modification treatment, amplification and high throughput sequencing, it is possible to analyze the methylation and/or hydroxymethylation status of at least 1000 or 5000 or more CpG islands in parallel.

A “CpG island” as used herein refers to regions of DNA with a high G/C content and a high frequency of CpG dinucleotides relative to the whole genome of an organism of interest. Also used interchangeably in the art is the term “CG island.” The in “CpG island” refers to the phosphodiester bond between the cytosine and guanine nucleotides.

DNA may be isolated from an organism of interest, including, but not limited to eukaryotic organisms and prokaryotic organisms, preferably mammalian organisms, such as humans, mice, or rats.

The human genome reference sequence (NCBI Build 36.1 from March 2006; assembled parts of chromosomes only) has a length of 3,142,044,949 bp and contains 26,567 annotated CpG islands (CpGs) for a total length of 21,073,737 bp (0.67%). In certain aspects, a DNA sequence read hits a CpG if the read overlaps with the CpG by at least 50 bp.

The methodologies of the current disclosure take advantage of the selective chemical labeling of 5hmC and a highly efficient transposase-based strategy. The methods of the disclosure generally include the following steps: a. modifying the 5hmC nucleic acid base with a first functional group; b. covalently attaching a modified nucleic acid probe comprising a second functional group to the first functional group; wherein the nucleic acid probe and nucleic acid molecule are covalently linked through the first and second functional groups; c. annealing a primer to the nucleic acid probe; d. performing primer extension of the annealed primer to make a new strand; and e. detecting the new strand. In the case of 5mC detection, endogenous 5hmC is first protected by attaching a non-functionalized molecule and then oxidizing 5mC to 5hmC. The steps a-e, as outlined above, are then performed.

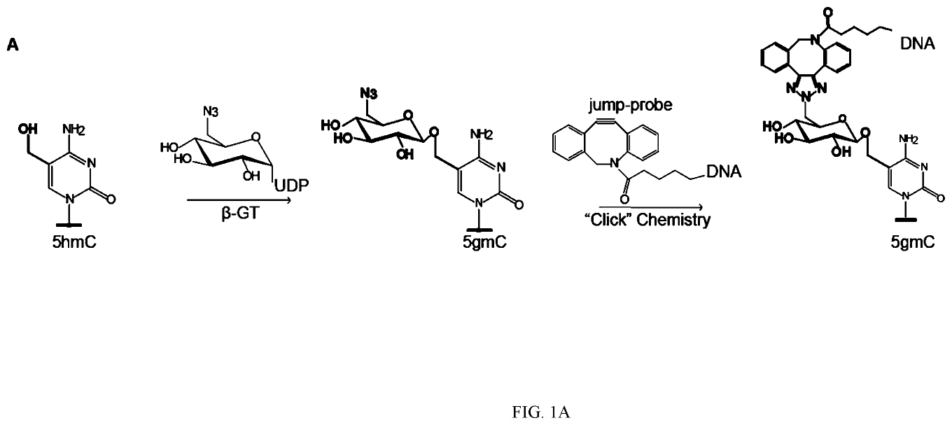

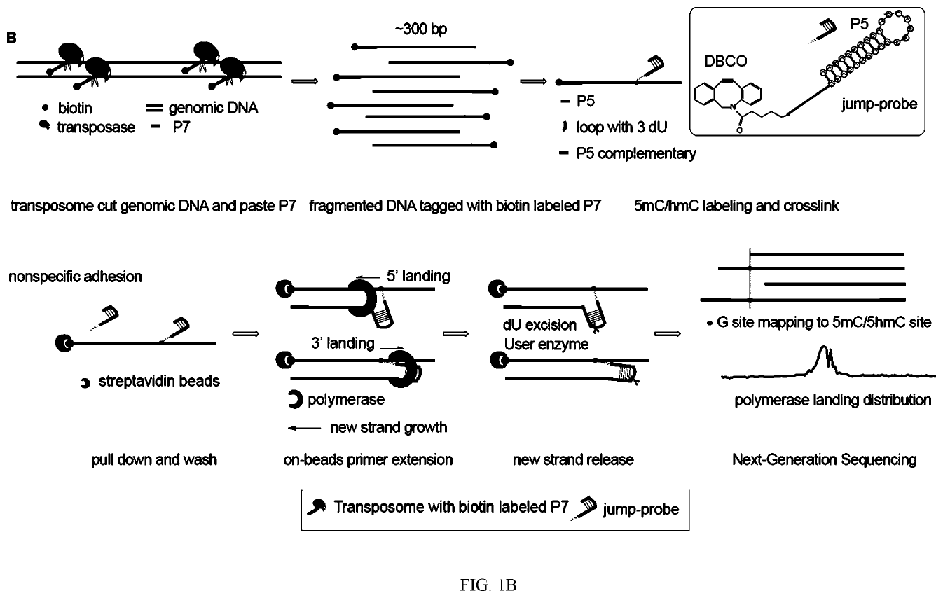

Shown in FIG. 1 is on embodiment in which genomic DNA was fragmented and tagged using transposome-based P7 adapter sequence (5′ Biotin-GTCTCGTGGGCTCGGAGATGTGTATAAGAGACAG 3′ (SEQ ID NO:5)); next, 5hmC was then labeled with a modified azide glucose utilizing βGT-mediated selective chemical labeling. Then, a hairpin DNA oligonucleotide with P5 adapter sequence and a unique sequence carrying an alkyne group was covalently connected to the azide-modified 5hmC. The loop part carries three deoxyribose uracils by design (5′ DBCO-CGAGTCANNNNNNNNCTGTCTCTTATACACATCTGACGCTGCCGdUdUdUTCGTC GGCAGCGTC 3′ (SEQ ID NO:6)). Next, primer extension from the hairpin DNA attached to 5hmC was run as indicated. The primer extension from the hairpin motif extends to the modified 5hmC site and will continue to “land” on the genomic DNA and reach the P7 adapter installed by transposase. The dU linker in the hairpin motif tethered to 5hmC was then cleaved by using USER™ enzyme. The extension products with P5 and P7 adapters were subsequently amplified and sequenced. 5mC/5hmC single sites were inferred from the “landing” site pattern that connects the hairpin sequence and any genomic DNA sequence.

The “landing” site pattern can be determined according to the following description. For each 50-bp Illumina sequencing read, fastx-trimmer was used to trim the first 8 bases which constitute a unique molecular identifier (UMI). The UMI sequence of each read was used later to remove PCR duplicates (reads starting at a same genomic location and sharing a same UMI sequence are likely to arise from one DNA fragment with a hydroxymethylated site, thus need to be collapsed and counted as one read). After extracting UMI, cutadapt (program available commercialy through PYTHON™) was used to retain reads with a Jump-seq barcode “TGACTCG” and to trim the barcode from each of these retained reads. Then the program bowtie (available for download online) was used to map the 35-bp reads to the relevant genome with default parameters. Only uniquely mapped reads were kept and processed with umi tools to remove PCR duplicates based on UMI sequences.

Using 5hmC sites in mouse ES cells identified by Tab-seq as examples. These sites were used as references to study the distribution of distance between Jump-seq read 5′ ends and 5hmC sites detected by Jump-seq. For plus-strand 5hmC sites, the distance distribution was plotted using reads aligning to minus-strand. For minus-strand 5hmC sites, reads aligning to plus-strand were used. To disentangle Jump-seq signals from single 5hmC sites, each 5hmC site was extended 100 bps both ways and only those extended intervals that don't overlap with others were used for calculating reads coverage. Reads coverage (5′ position) for each 5hmC-containing 201 bp interval was calculated by bedtools and added up across all intervals.

Around 5mc sites, the distribution of 5mC Jump-seq reads could plotted in the same manner as around 5hmC sites. Strand-unspecific 5mC sites were used as references for plotting 5′ ends of 5mc Jump-seq reads.

Suppose to look at one region (it could be the whole genome if it is large enough). Assuming there are K cytosines or C whose relative 5hmC level are θk, k=1,2, . . . ,K. θk specifies the normalized relative abundance of 5hmC at site k. The idea behind is each C has certain amount of chance of being hydroxylmethylated. The relative abundance involves much richer information than absolute enrichment determined mainly by number of reads.

The abundance level is characterized with the profiling of reads. Assume there are I reads in total with Ri indexing the i-th read. Let Ci denote the source 5hmC generating read Ri. So Ci is a latent variable and could be any possible site of K sites. Θk=P(Ci=k). Set Ci=0,1,2, . . . ,K with Ci=0 meaning read Ri is generated not from any cytosines which is a “noisy” read. Si denotes the distance of its start position to source site Ci, Si=0,1, . . . ,J. The empirical distribution of start positions of reads shows the bi-mode pattern which may not be symmetric, with the true 5hmC being in the “valley” between the two modes. These motivate the use of multinomial distribution to model the distribution of start positions with distance to the source 5hmC. Assume P (Sᵢ=j|Cᵢ)=πⱼ such that πⱼ≥0, Σπⱼ=1. In fact, the distribution of start position of ONEREAD is a categorical distribution with probability mass function of

$$P ( {S}_{i} \vert {C}_{i} ) = {\prod }_{j} {π}_{j}^{[ {S}_{i} = j ]}$$

This says that how the start sites are located only depends on the distance, not on the site i. The observed data are start positions of all reads. The interest is on the inference of Ok. For the noisy read, it is assumed to be uniformly distributed as

$$P ( {S}_{i} \vert {C}_{i} = 0 ) = \frac{1}{J + 1}$$

Let R=(R1, . . . ,RI) denotes all reads sample, π=(π0, . . . ,πJ), θ=(θ0,θ1, . . . ,θK). Assuming independence in generating the reads, the observed data likelihood function is

$$\begin{matrix}L ( π \vert R ) = {\prod }_{i} P ( {R}_{i} \vert π ) \\ = {\prod }_{i} {\sum }_{{C}_{i}} P ( {R}_{i} , {C}_{i} \vert π ) \\ = {\prod }_{i} {\sum }_{k} P ( {J}_{i} \vert {C}_{i} = k , π ) P ( {C}_{i} = k \vert π ) \\ = {\prod }_{i} {\sum }_{k} {θ}_{k} {\prod }_{j} {π}_{j}^{[ {S}_{i} = j ]}\end{matrix}$$

We use EM algorithm to find the Maximum Likelihood Estimate (MLE) of parameter θk. Use binary variable Zik=1 to indicate that reads i is from k-th 5hmC and Zik=0 otherwise. The complete likelihood is

$$\begin{matrix}P ( R , Z \vert π , θ ) = P ( R \vert Z , π , θ ) \times P ( Z \vert π , θ ) \\ = {\prod }_{i} {\prod }_{k} P ( {R}_{i} \vert {Z}_{ik} , π , θ ) \times P ( {Z}_{ik} \vert π , θ ) \\ = {\prod }_{i} {\prod }_{k} {{θ}_{k}^{{Z}_{ik}} ( 1 - {θ}_{k} )}^{1 - {Z}_{ik}} {\prod }_{j} {π}_{j}^{[ {S}_{i} = j ]}\end{matrix}$$

The EM algorithm consists of two steps, E step and M step:

E step: suppose parameter estimates at current step are θ(t),π(t), the Q function is

$$\begin{matrix}Q ( π , θ \vert {π}^{( t )} , {θ}^{( t )} ) = {E}_{Z \vert R , {π}^{( t )} , {θ}^{( t )}} log P ( R , Z \vert π , θ ) \\ = {\sum }_{i} {\sum }_{k} \{ E ( {Z}_{ik} \vert R , {π}^{( t )} , {θ}^{( t )} ) log ( {θ}_{k} ) + \\ ( 1 - E ( {Z}_{ik} \vert R , {π}^{( t )} , {θ}^{( t )} ) ) log ( 1 - \\ {θ}_{k} ) \} {\sum }_{j} [ {S}_{i} = j ] log ( {π}_{j} )\end{matrix}$$

$$And$$

$$\begin{matrix}E ( {Z}_{ik} \vert R , {π}^{( t )} , {θ}^{( t )} ) = P \{ {Z}_{ik} = 1 \vert {R}_{i} , {π}^{( t )} , {θ}^{( t )} \} \\ = \frac{P ( {R}_{i} , {π}^{( t )} , {θ}^{( t )} , {Z}_{ik} = 1}{P ( {R}_{i} , {π}^{( t )} , {θ}^{( t )} )} \\ = \frac{P ( {Z}_{ik} = 1 \vert {θ}^{( t )} ) P ( {R}_{i} \vert {π}^{( t )} , {Z}_{ik} = 1 )}{{\sum }_{k} P ( {Z}_{ik} = 1 \vert {θ}^{( t )} ) P ( {R}_{i} \vert {π}^{( t )} , {Z}_{ik} = 1 )} \\ = \frac{{θ}_{k}^{( t )} {\prod }_{j} {π}_{j}^{{( t )}^{[ {S}_{i} = j ]}}}{{\sum }_{k} {θ}_{k}^{( t )} {\prod }_{j} {π}_{j}^{{( t )}^{[ {S}_{i} = j ]}}} \\ = \frac{{θ}_{k}^{( t )}}{{\sum }_{k} {θ}_{k}^{( t )}}\end{matrix}$$

M step: update θ, π by maximizing Q function. Introducing Lagrange multiplier to the Q function, taking derivatives and setting to zero yields

$${\hat{π}}_{j}^{( t + 1 )} = \frac{{N}_{j}}{I}$$

where Nj={Ri, i=1, . . . ,I|Si=j}, the number of read starting at j, and I is the total number of reads

$${θ}_{k}^{( t + 1 )} = \frac{1}{I} {\sum }_{i} E ( {Z}_{ik} \vert R , {π}^{( t )} , {θ}^{( t )} )$$

With estimates of parameter θ, we have knowledge on which sites are very likely to be hydroxylmethylated and which are not.

This method relies on direct 5mC/5hmC capture, primer extension and amplification, which is streamlined, highly efficient and can potentially amplify even a few 5mC/5hmCs.

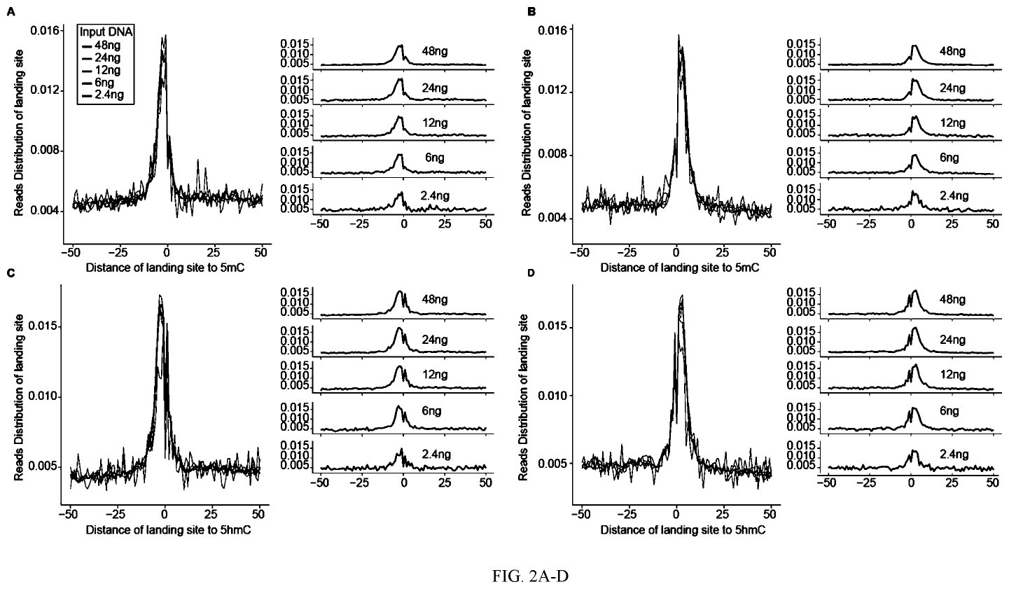

Applying the methods of the disclosure to genomic DNA from mouse ESCs (FIG. 2) has confirmed that this method can reveal base-resolution information of 5hmC. A unique distribution of the primer extension to the genomic DNA sequence was observed with the first encounter or “landing” sites distributed around the examined 5hmC sites and a “valley” overlaid on top of the 5hmC sites (FIG. 2C and FIG. 2D). A mechanistic explanation for this interesting “valley” formation is based on a potential differential behavior of the polymerases at the encounter of the “gap” (composed of the azide glucose and DBCO linker) between the unique DNA sequence attached to 5hmC and genomic DNA. The polymerase could overcome the obstacle and jump to genomic DNA to continue extension with high efficiency. During this jump some polymerases land 1˜14 bases 5′ ahead of the 5hmC site and continue to extend the strand, while others slide back to the genomic strand (-1˜-3 base towards the 3′) and then extend on the genomic template. Less polymerases land exactly on the modified 5hmC sites, thus forming a “valley” at the exact 5hmC site.

In addition, as the double-stranded DNA strands have been denatured into single-stranded before attachment of the nucleic acid probe, and the “click” based crosslink is efficient and unbiased, the methods of the disclosure can clearly reveal the precise positions of 5hmCs on the Watson and Crick strands of fully-hydroxymethylated hmCpGs (FIG. 2), demonstrating the single-base accuracy. The 5mC data of mouse ESCs genomic DNA also reveal optimal overlap of 5mC loci with sites identified by TAB-seq (FIGS. 2A and 2B).

B. Base-Resolution Sequencing of 5mC and 5hmC in Single Cell Level.

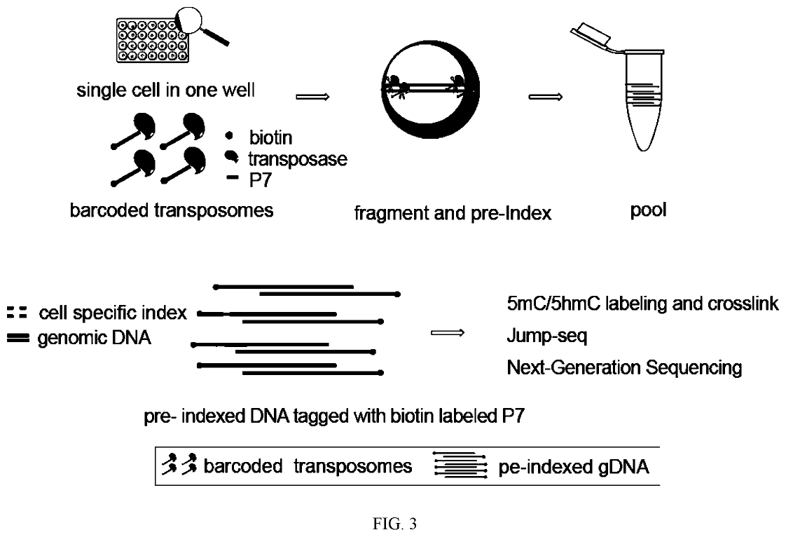

Flow cytometry is frequently used for isolation and identification of single cells, since different subpopulations are characterized by the existence of specific combinations of surface markers. Based on the multicolored fluorescence-assisted cell sorting (FACS) using monoclonal antibodies, a series of single-cell new methods have been developed, resulting in: i) detection of proteins in single cell by coupling with mass spectrometry, ii) investigation of single-cell transcriptional programs by coupling with RNA-seq and iii) profiling chromatin signature by coupling with Chip-seq. The methods of the disclosure can be used to develop a streamlined technology that combine single cell sorting, DNA barcoding, and 5mC/5hmC Jump-seq strategy to map 5mC and 5hmC at single cell level and base resolution (FIG. 3). To achieve single-cell pre-index barcoded transposomes carrying cell specific barcodes are used. First, targeted cells were sorted into 384 well plates by flow cytometry, followed by adding barcoded transposomes. Each cell receives one specific transposome carrying a unique barcode.

After each cell is barcoded, the tagged genomic DNA fragments are combined for 5hmC (or 5mC) nucleic acid probe attachment, primer extension, library construction, and subsequent sequencing. As 5mC/5hmC jump-products from each cell carry a unique barcode, 5mC/5hmC reads from each individual cell can be computationally separated.

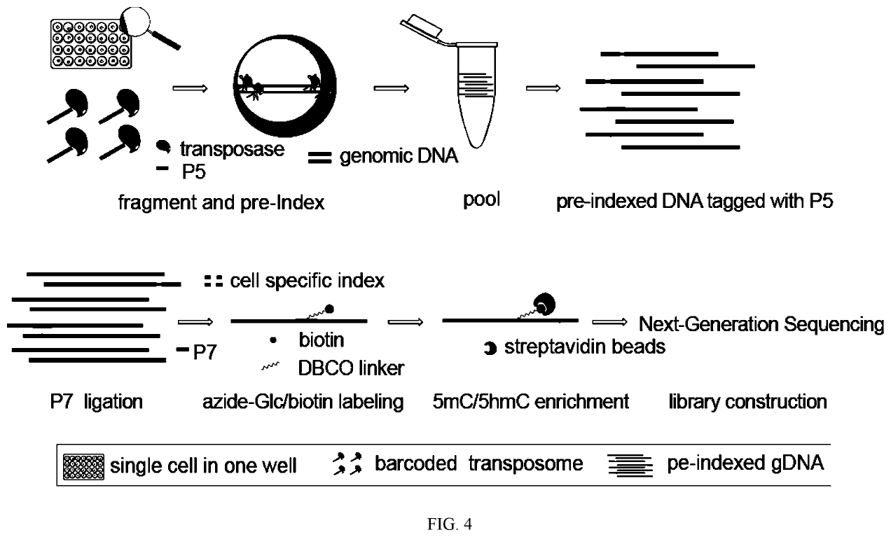

In an alternative approach, single cell mC/hmC-Seal method can be used to validate mC/hmC distribution identified by the methods of the disclosure (FIG. 4). Briefly, single hematopoietic cells are sorted into 384 well plate in one-cell-one-well manner, then transposome assembled with cell specific barcodes is added to the wells (a unique barcoded transposome is added to each individual well) to pre-index genomic DNA. Next, the indexed genomic DNA is pooled, followed by the well established 5mC/5hmC-Seal method known in the art (see, for example, WO/2012/138973, which is herein incorporated by reference) to enrich and pull down 5mC/5hmC-containing DNA fragments. The single-cell mC/hmC-Seal method and single cell 5mC/5hmC methods of the disclosure will serve as fail-safe to subtly map hematopoietic methylome and hydroxymethylome landscape.

C. Detection of 5mC/5hmC in Cell Free DNA.

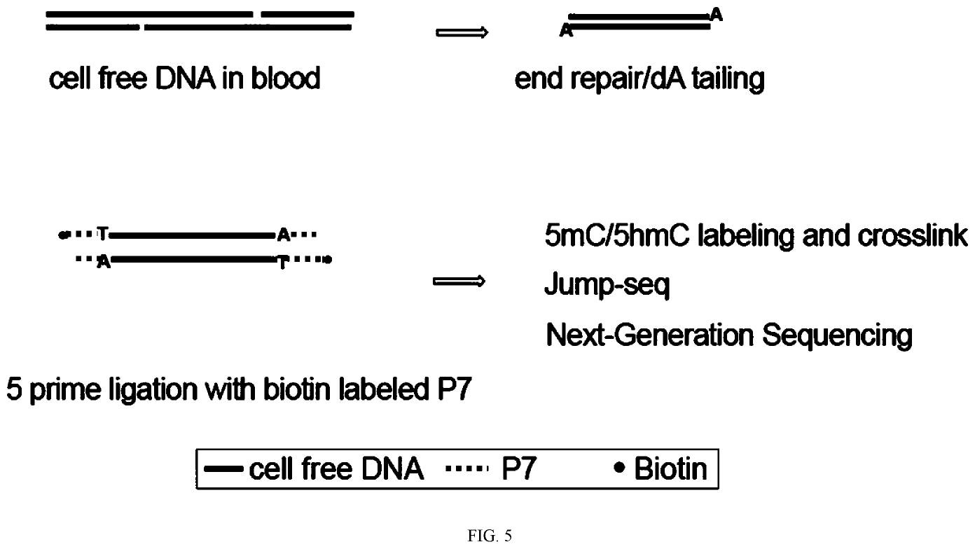

Cell-free DNA, the double stranded and highly fragmented molecules with 100 bp-400 bp in length, is detectable in circulating blood and has the clinical potential to be a more specific tumor marker for the diagnosis and prognosis, as well as the early detection of cancer. Fetal DNA circulating freely in the maternal blood stream can be sampled by venipuncture on the mother. Analysis of cell-free fetal DNA provides a method of non-invasive prenatal diagnosis and testing. The methods of the disclosure can be used to perform 5mC/5hmC profiling in cell free DNA with a streamlined flowchart: Cell free DNA is end repaired, ligated with P7 at the 5′ end, followed by application of the methods of the disclosure (FIG. 5).

D. Jump-qPCR and Jump-Array

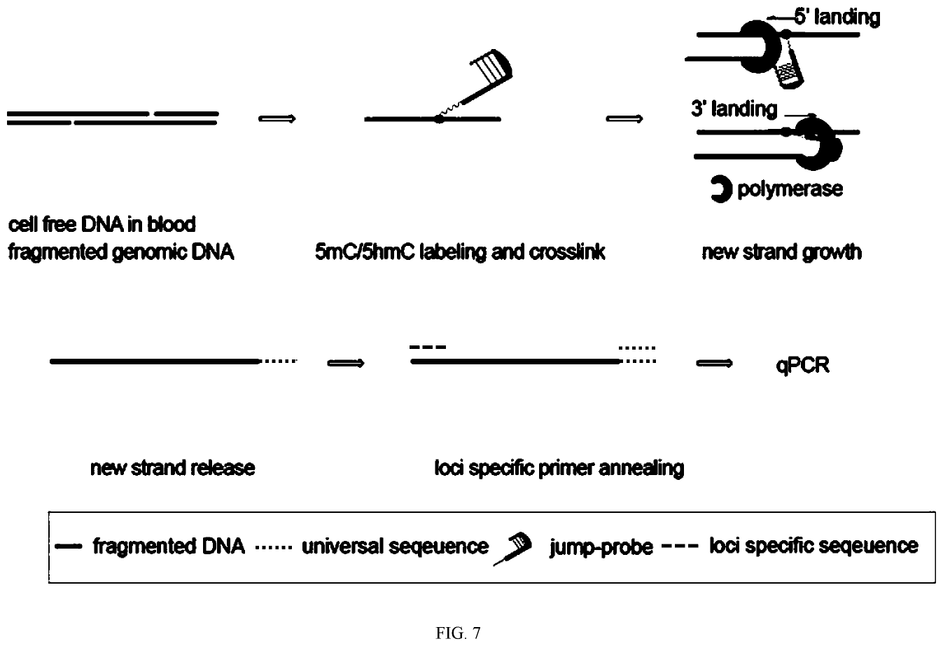

As shown in FIG. 7, the current methods of the disclosure can be used for a Jump-qPCR method in which specific loci are detected using a universal primer that binds to the primer annealed/attached to the probe and a loci-specific primer. The specific loci then may be detected by methods known in the art such as sequencing or by quantitative PCR.

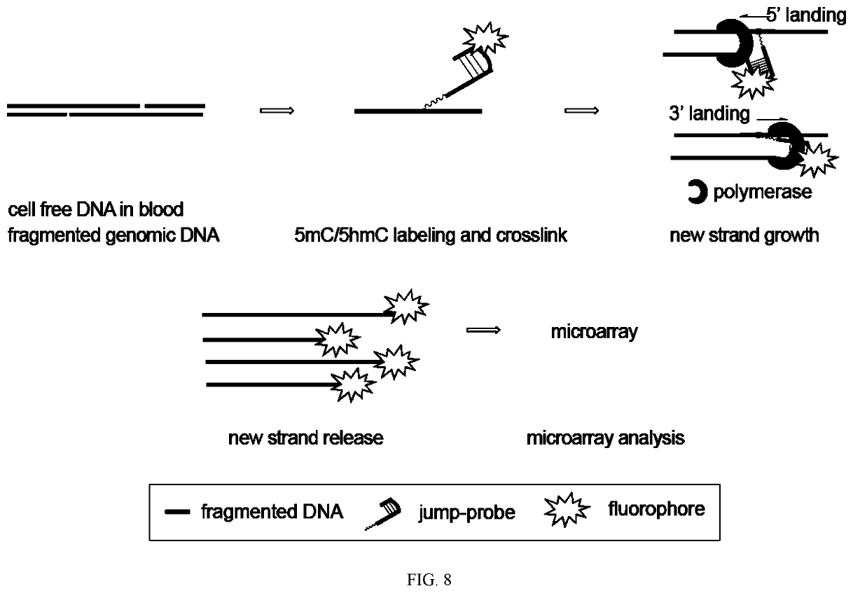

As shown in FIG. 8, the current methods of the disclosure can be used for a Jump-array method in which the newly synthesized fluorescent strands are subjected to a microarray.

If a number (tens) of 5hmC and 5mC sites/loci have already been identified through Jump-seq, 5hmC-Seal/5mC-Seal or related method for a specific cancer or disease or test, high-throughput sequencing could be a bit costly, however, qPCR and microarray are practical and cheaper alternatives.

For Jump-qPCR, the cell free DNA or fragmented DNA can be crosslinked with jump-probe that contains a specific universal sequence followed by primer extension. The released newly synthesized strands were annealed with designed loci specific primer and subjected to qPCR. Jump-qPCR is a very useful method for quantitative assessment of 5hmC/5mC amount at specific loci (detecting a few to tens of sites).

For Jump-array, the procedure is mainly the same except that the jump-probe contains a fluorophore so that the released newly synthesized fluorescent strands could be subjected to microarray fluorescent scan.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for detecting 5-hydroxymethylcytosine (5hmC) nucleic acid bases in a nucleic acid molecule or a plurality of nucleic acid molecules, the method comprising:
a. modifying the 5hmC nucleic acid base with a first functional group;
b. covalently attaching a modified nucleic acid probe comprising a second functional group to the first functional group; wherein the nucleic acid probe and nucleic acid molecule are covalently linked through the first and second functional groups;
c. annealing a primer to the nucleic acid probe;
d. performing primer extension of the annealed primer to make a new strand; and
e. detecting the new strand.

2. The method of claim 1, wherein detecting the new strand comprises sequencing the new strand and/or polymerase chain reaction.

3. (canceled)

4. The method of claim, wherein the primer and/or probe is labeled with a detection moiety and further wherein detecting the new strand comprises detecting the detection moiety.

5-6. (canceled)

7. The method of claim 1, wherein the nucleic acid molecule comprises genomic DNA.

8. (canceled)

9. The method of claim, wherein the first functional group is covalently attached to a glucose or a modified glucose molecule.

10. The method of claim 1, wherein the 5hmC is modified with a glucose or a modified glucose molecule and wherein modifying the 5hmC nucleic acid base with a glucose or a modified glucose comprises incubating the nucleic acid molecule with a β-glucosyltransferase and a glucose or modified glucose molecule.

11. (canceled)

12. The method of claim 10, wherein the modified glucose molecule is uridine diphospo6-N₃-glucose molecule.

13. (canceled)

14. The method of claim 1, wherein the first or second functional groups comprise an alkyne, azide, thiol, or maleimide.

15-16. (canceled)

17. The method of claim 1, wherein the nucleic acid probe is modified with a molecule having a molecular mass of at least 150 u.

18-22. (canceled)

23. The method of claim 1, wherein the nucleic acid is tagged and/or fragmented by a transposome wherein tagging and/or fragmenting the nucleic acid comprises contacting the contacting the nucleic acid molecule with a transposase and a transposon.

24. (canceled)

25. The method of claim 23, wherein the transposon comprises a P7 adapter-containing transposon and/or an affinity tag.

26-27. (canceled)

28. The method of claim 25, wherein the method further comprises isolating or purifying the fragmented nucleic acid molecules by contacting the nucleic acid molecules with a capture reagent, wherein the capture reagent binds to the affinity tag; and separating the capture reagent bound to the affinity tagged fragmented nucleic acid molecules from surrounding components.

29. The method of claim 1, wherein the method further comprises sorting a population of cells into isolated single cells and wherein the method further comprises tagging the nucleic acid of each single cell with a unique nucleic acid sequence.

30. (canceled)

31. The method of claim 29, wherein the method further comprises pooling the tagged nucleic acids into a single composition.

32. The method of claim 1, wherein the nucleic acid comprises cell free DNA and wherein the cell-free DNA is isolated from the blood.

33-36. (canceled)

37. The method of claim 1, wherein the probe comprises a cleavage site.

38. The method of claim 1, wherein the nucleic acid probe comprises a hairpin and optionally wherein the hairpin comprises a loop comprising deoxyribose uracils.

39-40. (canceled)

41. The method of claim 38, wherein the method further comprises cleaving the loop with a uracil DNA glycosylase.

42-50. (canceled)

51. The method of claim 1, wherein the nucleic acid molecule or molecules is present in an amount of less than 50 ng.

52-54. (canceled)

55. A method for detecting 5-methylcytosine (5-mC) nucleic acid bases in a nucleic acid molecule or a plurality of nucleic acid molecules, the method comprising:
a. modifying 5-hmC nucleic acid bases with a glucose molecule;
b. oxidizing 5-mC to 5-hmC to make converted 5-hmC;
c. modifying the converted 5-hmC nucleic acid base with a first functional group;
d. covalently attaching a modified nucleic acid probe comprising a second functional group to the first functional group; wherein the nucleic acid probe and nucleic acid molecule are covalently linked through the first and second functional groups;
e. annealing a primer to the nucleic acid probe;
f. performing primer extension of the annealed primer to make a new strand; and
g. detecting the new strand.

56-109. (canceled)


SEQUENCE LISTING (6 sequences)

SEQ ID NO: 1 - protein, 375 aa, Homo sapiens
SHHHHHHSSGVDLGTENLYFQSNAMKIAIINMGNNVINFKTVPSSETIYLFKVISEMGLNVDIISLKNGVYTKSFDEVDVNDYDRLIVVNSSINFFGGKPNLAILSAQKFMAKYKSKIYYLFTDIRLPFSQSWPNVKNRPWAYLYTEEELLIKSPIKVISQGINLDIAKAAHKKVDNVIEFEYFPIEQYKIHMNDFQLSKPTKKTLDVIYGGSFRSGQRESKMVEFLFDTGLNIEFFGNAREKQFKNPKYPWTKAPVFTGKIPMNMVSEKNSQAIAALIIGDKNYNDNFITLRVWETMASDAVMLIDEEFDTKHRIINDARFYVNNRAELIDRVNELKHSDVLRKEMLSIQHDILNKTRAKKAEWQDAFKKAIDL

SEQ ID NO: 2 - DNA, 34 bp, Artificial Sequence
  Synthetic Adapter
gtctcgtgggctcggagatgtgtataagagacag

SEQ ID NO: 3 - DNA, 13 bp, Artificial Sequence
  Synthetic Primer
cgtcggcagcgtc

SEQ ID NO: 4 - DNA, 61 bp, Artificial Sequence
  Synthetic Probe
  misc_feature (8)..(15): n is a, c, g, t or u
  misc_feature (45)..(47): U is d configuration
cgagtcannnnnnnnctgtctcttatacacatctgacgctgccguuutcgtcggcagcgtc

SEQ ID NO: 5 - DNA, 34 bp, Artificial Sequence; identical to SEQ ID NO: 2
  Synthetic Adapter
  misc_feature (1)..(1): 5′ biotin tagged

SEQ ID NO: 6 - DNA, 61 bp, Artificial Sequence; identical to SEQ ID NO: 4
  Synthetic Adapter
  misc_feature (1)..(1): 5′ DBCO tagged
  misc_feature (8)..(15): n is a, c, g, t or u
  misc_feature (45)..(47): U is d configuration